(19) United States
(12) Patent Application Publication
(10) Pub. No.: US 2026/0027190 A1
(43) Pub. Date: Jan. 29, 2026

(54) OVARIAN TUMOR DEUBIQUITINASE OXIDATION AND USES THEREOF

(71) Applicant: The University of Vermont and State Agricultural College, Burlington, VT (US)

(72) Inventors: Yvonne M. Janssen-Heininger, Charlotte, VT (US); Reem Aboushousha, South Burlington, VT (US); Jos van der Velden, South Burlington, VT (US); Jianing Li, West Lafayette, IN (US)

(73) Assignee: The University of Vermont and State Agricultural College, Burlington, VT (US)

(21) Appl. No.: 18/993,082

(22) PCT Filed: Jul. 10, 2023
(86) PCT No.: PCT/US2023/069911

§ 371 Date: Jan. 10, 2025

Related U.S. Application Data
(60) Provisional application No. 63/388,203, filed on Jul. 11, 2022.

(51) Int. Cl. A61K 38/44; A61K 31/454; A61K 31/4725; A61K 31/496; A61K 33/243; A61P 35/00

(52) U.S. Cl. CPC A61K 38/443; A61K 31/454; A61K 31/4725; A61K 31/496; A61K 33/243; A61P 35/00; C12Y 111/01009

(57) ABSTRACT

Disclosed herein are compositions comprising agents that inhibit or prevent the oxidation of one or more cysteine resides in ovarian tumor deubiquitinase (OTUB1) as well as methods of using the compositions, for example, to treat cancers (e.g., lung cancers). Also provided herein are kits comprising the agents.

### RELATED APPLICATIONS

This application claims the benefit of U.S. Provisional Patent Application Ser. No. 63/388,203, filed Jul. 11, 2022, entitled “Ovarian Tumor Deubiquitinase Oxidation and Uses Thereof,” by Janssen-Heininger, et al., incorporated herein by reference in its entirety.

### GOVERNMENT FUNDING

This invention was made with government support under HL135828 and CA219156 awarded by the National Institutes of Health. The government has certain rights in the invention.

### REFERENCE TO AN ELECTRONIC SEQUENCE LISTING

The contents of the electronic sequence listing (V013970134WO00-SEQ-TC.xml; Size: 15,459 bytes; and Date of Creation: Jul. 10, 2023) is herein incorporated by reference in its entirety.

### BACKGROUND

Oxidants are key regulators of certain physiological processes, and the highly abundant tripeptide molecule glutathione (GSH) is one of the major antioxidants that controls cellular redox homeostasis. GSH plays important roles in the function of regulatory T-lymphocytes, activation of epithelial cells and survival of cancer cells, among others. Glutathione is a tripeptide of cysteine, glycine and glutamic acid, and its synthesis occurs in a series of steps that involves the formation of gamma glutamyl-cysteine via glutamate cysteine ligase (GCL), followed by the addition of glycine via glutathione synthase (GS). Levels of glutathione are tightly controlled via negative feedback regulation of the catalytic subunit of GCL and degradation of GSH via a series of enzymes.

### SUMMARY

The disclosure, in some aspects, provides a method of treating a cancer, the method comprising administering an effective amount of an agent to a subject in need thereof, wherein the agent inhibits the oxidation under physiological conditions of a cysteine residue of ovarian tumor deubiquitinase (OTUB1).

In some embodiments, the cysteine residue of OTUB1 comprises Cys23. In some embodiments, the cysteine residue of OTUB1 comprises Cys204.

In some embodiments, the agent inhibits the oxidation of a second cysteine residue of OTUB1. In some embodiments, the agent inhibits the cysteine residue and the second cysteine residue comprise Cys23 and Cys204.

In some embodiments, the cancer is selected from the group consisting of: breast cancer, ovarian cancer, lung cancer, liver cancer, colon cancer, glioma, melanoma, acute myeloid leukemia, esophageal cancer, gastric cancer, endometrial cancer, prostate cancer, and thyroid cancer. In some embodiments, the lung cancer is selected from the group consisting of: non-small cell lung cancer, squamous cell carcinoma, adenocarcinoma, large-cell carcinoma and small-cell lung cancer.

In some embodiments, the agent comprises a small molecule. In some embodiments, the agent comprises a glutaredoxin. In some embodiments, the glutaredoxin is selected from the group consisting of glutaredoxin 1 (GRX1), GRX2, and GRX5.

In some embodiments, the subject has a cancer.

In some embodiments, the method further comprises reducing a glutathione level in a cancer cell, destabilizing Solute Carrier Family 7 Member 11 (SLC7A11), decreasing the function of system xC- or any combination thereof. In some embodiments, the method further comprises concomitantly administering an additional therapeutic agent. In some embodiments, the additional therapeutic agent is an immune checkpoint inhibitor or a chemotherapeutic agent. In some embodiments, the immune checkpoint inhibitor is selected from the group consisting of: CTLA4, PD1, PDL-1, B7H1, B7H3, B7H4, OX-40, CD137, CD40, CD27, LAG3, TIM3, ICOS, or BTLA. In some embodiments, the chemotherapeutic agent comprises cisplatin.

The disclosure, in some aspects, provides an agent that inhibits the oxidation of at least one cysteine residue of ovarian tumor deubiquitinase (OTUB1) under physiological conditions.

In some embodiments, the agent inhibits the oxidation of a second cysteine residue of OTUB1. In some embodiments, the agent inhibits the cysteine residue and the second cysteine residue comprise Cys23 and Cys204

In some embodiments, the agent comprises a small molecule. In some embodiments, the agent comprises a glutaredoxin. In some embodiments, the glutaredoxin includes (but is not limited to) one or more of glutaredoxin 1 (GRX1), GRX2, and GRX5.

In some embodiments, the disclosure provides a pharmaceutical composition comprising any one of the agents described herein and a pharmaceutically acceptable excipient.

The disclosure provides, in some aspects, a kit comprising a container housing an agent that inhibits the oxidation of at least one cysteine residue of ovarian tumor deubiquitinase (OTUB1) and instructions for administering components in the kit to a subject having a cancer. In some embodiments, the kit further comprises a container housing a pharmaceutical preparation diluent.

The subject matter of the present disclosure involves, in some cases, interrelated products, alternative solutions to a particular problem, and/or a plurality of different uses of one or more systems and/or articles. It is anticipated that each of the limitations described herein involving any one element or combinations of elements can be included in other aspects. This disclosure is not limited in its application to the details of construction and the arrangement of components set forth in the following description or illustrated in the drawings. The disclosure is capable of other embodiments and of being practiced or of being carried out in various ways. The details of one or more embodiments are set forth in the accompanying Detailed Description, Examples, and Claims. Other features, objects, and advantages will be apparent from the description and from the claims.

### BRIEF DESCRIPTION OF THE DRAWINGS

The accompanying drawings are not intended to be drawn to scale. In the drawings, each identical or nearly identical component that is illustrated in various figures is represented by like numeral. For purposes of clarity, not every component may be labeled in every drawing. In the drawings:

FIG. 1 is a schematic representing the system xC- mechanism of regulation whereby glutathione-dependent protein oxidation may be under glutaredoxin-mediated control, in one embodiment of the present disclosure.

FIGS. 2A-21 demonstrate that interleukin 1B (IL1B) increases GSH and SLC7A11 levels in mouse tracheal epithelial cells.

FIGS. 3A-3D demonstrate the formation of an oxidation-dependent high molecular weight (HMW) complex of SLC7A11, SLC3A2 and OTUB1 in response to stimulation with IL1B.

FIGS. 4A-4L demonstrate S-glutathionylation regulation of GSH in an xC-/OTUB1-dependent manner.

FIGS. 5A-5J demonstrate the interaction of SLC7A11, OTUB1 and SLC3A2 in H522 lung adenocarcinoma cells and show that Glrx and OTUB1 may regulate SLC7A11 and GSH.

FIGS. 6A-6E demonstrate that S-glutathionylation at Cys23 and Cys204 of OTUB1 stabilizes system xC- and increases GSH levels in a system xC- dependent manner.

FIGS. 7A-7G demonstrate that S-glutathionylation of OTUB1 at Cys23 and Cys204 decreases SLC7A11 ubiquitination and promotes the SLC7A11 and SLC3A2 interaction. The figures also show a role of the proteasome in the degradation of SLC7A11 and subsequent decreases in GSH.

FIG. 8 demonstrates molecular modeling visualizing the impact of OTUB1-SSG on SLC7A11 and SLC3A2 interactions.

FIGS. 9A-9B demonstrate that carbons from glucose are used in synthesis of GSH.

FIGS. 10A-10D demonstrate mechanisms whereby IL1B increases GSH.

FIG. 11 shows the effects of inhibiting system xC- on IL1B-induced pro-inflammatory cytokines.

FIGS. 12A-12D demonstrate S-glutathionylation at Cys23 and Cys204 of OTUB1 stabilizes system xC- and increases GSH levels in OTUB1-CRISPR H522 cells.

FIGS. 13A-13C demonstrate that GLRX increases cisplatin induced killing in association with decreasing GSH.

FIGS. 14A-14H demonstrate how the E2 conjugating enzyme, UBCH5A interacts with OTUB1 in a GLRX-sensitive manner to increase SLC7A11 and GSH.

FIGS. 15A-15D show molecular modeling visualizing the impact of OTUB1-SSG at C23 or C204 on the binding between OTUB1 and UBCH5A.

FIGS. 16A-16B demonstrate the altered expression of GLRX, SLC7A11 and OTUB1 in LUAD.

FIGS. 16C-16I demonstrate the increased PSSG, GSH, OTUB1-SSG and SLC7A11 in LUAD.

### DETAILED DESCRIPTION

Without further elaboration, it is believed that one skilled in the art can, based on the description herein, utilize the present disclosure to its fullest extent. The following specific embodiments are, therefore, to be construed as merely illustrative, and not limitative of the remainder of the disclosure in any way whatsoever.

The disclosure relates, in some embodiments, to the discovery of two amino acid residues of an enzyme that are involved in improving the function of a pathway that is active in numerous cancers. Therefore, without wishing to be bound by theory, it is thought that preventing the oxidation of one or both of the amino acids identified could destabilize the pathway, improving tumor killing and the efficacy of certain anti-cancer therapeutics.

In particular, provided herein are methods of preventing or inhibiting the S-glutathionylation oxidation of one or both amino acids, Cys23 and/or Cys204, of ovarian tumor deubiquitinase, OTUB1, thereby reducing the function of system xC- and destabilizing SLC7A11 (XCT). Without wishing to be bound by theory, it is thought that inhibition of the oxidation of OTUB1 Cys23 and/or Cys204 destabilizes SLC7A11 and system xC-, such that extracellular cystine is not converted to cysteine and transported into the cell (e.g., cancer cell). Cysteine starvation may lead to GSH depletion in the cell and eventual cell death, e.g., through ferroptosis. Thus, the agents provided herein may provide improved tumor killing, an improved sensitivity to immune checkpoint inhibitors, and/or an increased sensitivity to chemotherapeutics (e.g., cisplatin).

### Ovarian Tumor Deubiquitinase (OTUB1) and Glutathione-Related Pathways

Described herein are compositions and agents that prevent or inhibit the oxidation of cysteines in ovarian tumor debiquitinase (OTUB1). OTUB1 (NCBI Gene ID 55611) also known as OTU domain-containing ubiquitin aldehyde-binding protein 1, OTU ubiquitin aldehyde binding 1, deubiquitinating enzyme OTUB1, and otubain-1 (hOTU1), is a deubiquitinating enzyme. Debiquitination regulates a variety of cellular functions, including apoptosis, cell signaling, and growth. OTUB1 inhibits cytokine gene transcription in the immune system by interacting with ubiquitin proteases and E3 ubiquitin ligase and is a highly specific ubiquitin iso-peptidase. Ubiquitin-binding occurs at the Cys91 active site, with proximal binding site present at Cys23. OTUB1 is the first known member of the OTU family proteins and is known to be uniformly distributed in all tissues. Unlike other deubiquitinating enzymes, OTUB1 can inhibit DNA damage in a non-catalytic manner by binding to the E2 enzyme UBC13 rather than directly deubiquitinating its substrate. OTUB1 may regulate deubiquitinating enzymes by binding to UbcH5, UBE2D, and UBE2E. In addition, OTUB1 may regulate DNA damage response, cell apoptosis, proliferation, and cancer development, as p53, SMAD2/3, and TRAF3 can be targets of OTUB1. OTUB1 also can control IL-15-stimulated activation of CD8+ T cells and natural killer (NK) cells. The deletion of OTUB1 may promote antitumor immunity, and OTUB1 may act as a checkpoint during T cell-mediated immune responses. Without wishing to be bound by theory, it is thought that, as OTUB1 modulates T cell function, highly expressed OTUB1 in cancer cells may regulate immune evasion. OTUB1 may positively regulate PD-L1 stability and mediate cancer immune responses through the PD-1/PD-L1 axis.

In addition, an S-glutathionylated proteome screen revealed that in lung epithelial cells stimulated with IL1B, OTUB1 was a target for PSSG. OTUB1 was shown to interact with SLC7A11, thereby enhancing its stability and augmenting system xC- activity in cancer cells.

SLC7A11 ((NC_005101.2), is a drug transporter protein. SLC7A11 forms a heteromultimeric complex with SLC3A2 which makes up the amino acid transport system, xCT. This amino acid transport system mediates cystine entry coupled exodus of glutamate and regulates intracellular glutathione levels. In some cases, gliomas may secrete glutamate via xCT which causes neuronal cell death. SLC7A11 may have a positive correlation with L-alanosine; increased levels of SLC7A11 expression lead to increased L-alanosine transport. SLC7A11 also may exhibit a negative correlation with certain drugs, and its control of glutathione levels contributes to the resistance of certain cancer drugs, such as cisplatin. SLC7A11 also may exhibit chemoresistance to multiple drugs and compounds.

System xC-, also known as the glutamate/cystine antiporter, is a transmembrane protein expressed in a variety of cells, including neural (e.g. astrocytes, microglia, immature cortical neurons and glioma cells) and non-neural (e.g. fibroblasts, macrophages, hepatocytes and endothelial) cells. System xC- may function as an antiporter/exchanger to import L-cystine into the cell and export L-glutamate out of the cell. The imported L-cystine within the cell may facilitate the production of GSH, the body's primary antioxidant, and the exported L-glutamate can act as an extracellular neurotransmitter. Due to its bimodality, system xC- may facilitate a wide range of central nervous system (“CNS”) functions, including oxidative protection, the operation of the blood-brain barrier, neurotransmitter release, synaptic organization and cyto-architecture, viral pathology, drug addiction, chemosensitivity, chemoresistance, and tumor growth within the brain as well as in peripheral compartments (e.g., breast and bladder).

Therefore, provided herein in certain embodiments are therapeutic agents that can inhibit or prevent the oxidation of one or both of Cys23 and Cys204 of OTUB1, thereby downregulating system xC- via destabilization of SLC7A11, and potentially killing cancer cells (e.g., through ferroptosis).

The sequence of OTUB1 is provided below (Cys23 and Cys204 are bolded and underlined):

(SEQ ID NO: 15)
MAAEEPQQQK QEPLGSDSEG VNCLAYDEAI MAQQDRIQQE
IAVQNPLVSE RLELSVLYKE YAEDDNIYQQ KIKDLHKKYS
YIRKTRPDGN CFYRAFGFSH LEALLDDSKE LQRFKAVSAK
SKEDLVSQGF TEFTIEDFHN TFMDLIEQVE KQTSVADLLA
SFNDQSTSDY LVVYLRLLTS GYLQRESKFF EHFIEGGRTV
KEFCQQEVEP MCKESDHIHI IALAQALSVS IQVEYMDRGE
GGTTNPHIFP EGSEPKVYLL YRPGHYDILY K.

### Agents

Provided herein are therapeutic agents that can inhibit or prevent oxidation (e.g., via S-glutathionylation) of OTUB1 residues under physiological conditions (i.e., healthy or normal subject conditions, such as temperature and/or pH). As used herein, “oxidation” refers to any reaction that includes loss of electrons. An “oxidized” protein (e.g., OTUB1), as used herein, is a protein in which at least one (native) amino acid residue of the protein (e.g., cysteine) has been oxidized in some fashion, relative to its initial state after formation. As an example, glutathione may react with a residue on the protein to glutathionylate the residue. Thus, as used herein, a “glutathionylated” protein is a protein in which at least one amino acid residue of the protein has been glutathionylated, i.e., the amino acid residue has reacted with glutathione, typically through the addition of the glutathione (or a portion thereof) to the residue. Residues that may undergo reactions with glutathione include sulfhydryl moieties (—SH) (e.g., from a cysteine residue), hydroxyl moieties (—OH) (e.g., from a serine residue or a threonine residue), or the like. In some embodiments, the residue includes a sulfhydryl moiety (—SH) (also referred to as a thiol moiety), and reaction of the moiety with glutathione produces an S-glutathionylated moiety, i.e., —S—S-G, where “G” represents glutathione). The “S—” signifies reaction with the sulfhydryl moiety

In some embodiments, the agent inhibits or prevents oxidation (e.g., via S-glutathionylation) of Cys23 of OTUB1. In some embodiments, the agent inhibits or prevents oxidation (e.g., via S-glutathionylation) of Cys204 of OTUB1. In some embodiments, the agent inhibits or prevents oxidation (e.g., via S-glutathionylation) of Cys23 and Cys204 of OTUB1.

As used herein, “inhibit” means to decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level. A reference level, in some embodiments, is the amount of OTUB1 oxidation (and/or Cys23 and/or Cys204 oxidation) in the absence of the agent. “Prevent” refers to the suppression of any oxidation (i.e., 100%) with respect one or both of the OTUB1 cysteine residues. Oxidation may be measured by any technique known in the art, for example, by quantitative formation of a chromophore or fluorophore, liquid chromatography, gel electrophoresis, or mass spectrometry. In some embodiments, oxidation is measured as the loss of free thiol groups in proteins (micromolar thiol per mg protein) using Ellman's reagent (e.g., 5,5′-dithiobis-(2-nitrobenzoic) acid), for example with a commercially available assay (e.g., DTNB's Thiol Oxidative Stress Assay).

As described herein, inhibition of the oxidation of OTUB1 Cys23 and/or Cys204 may result in a reduction of glutathione expression levels (e.g., in cancer cells) and cell death through ferroptosis in certain embodiments. Therefore, the agents described herein may indirectly reduce glutathione expression levels in cells (e.g., cancer cells), destabilize SLC7A11, and/or decrease activity/function of system xC-, etc. Without wishing to be bound by theory, it is thought that inhibition of the oxidation of OTUB1 Cys23 and/or Cys204 destabilizes Solute Carrier Family 7 Member 11 (SLC7A11) and system xC-, such that extracellular cystine is not converted to cysteine and transported into the cell (e.g., cancer cell). Cysteine starvation can lead to GSH depletion in the cell and eventual cell death through ferroptosis.

In some embodiments, the agent comprises a small molecule. As used herein, “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

In some embodiments, the agent targets Cys23, for example, by binding OTUB1 near Cys23. In some embodiments, the small molecule is a non-covalent compound. The non-covalent docking score, a measurement of binding in the Cys23 region, in some embodiments, is at least −9 (e.g., −9.1, −9.2, −9.3, −9.4, −9.5, −9.6, −9.7, −9.8, −9.9, or −10). Non-covalent docking scores may be determined by any method in the art, for example with the Schrödinger platform. The covalent docking score, a measurement of binding in the Cys23 region, in some embodiments, is at least −4 (e.g., −4.1, −4.2, −4.3, −4.4, −4.5, −4.6. −4.7, −4.8, −4.9, −5.0, −5.25, −5.5, −5.75, −6.0, −6.1). Covalent docket scores may be obtained from any method known in the art, for example, using simulations with the Covalent Dock Lead Optimization (CovDock-LO) and Virtual Screening (CovDock-VS) Workflows with the Schrödinger platform. Exemplary non-covalent small molecule inhibitors that target Cys23 of OTUB1 are as follows:

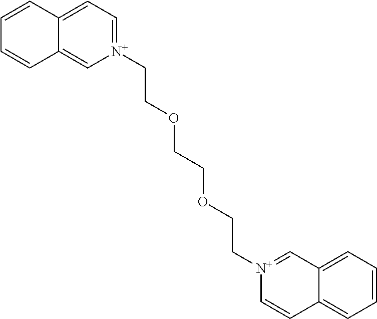 [SMILES: c1ccc2c[n+](CCOCCOCC[n+]3ccc4ccccc4c3)ccc2c1]

(non-covalent docking score, −9.379; covalent docking score, −4.529).

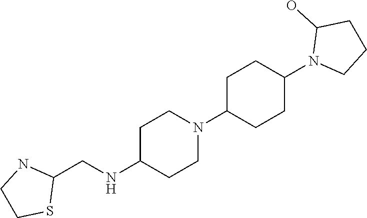 [SMILES: OC1CCCN1C1CCC(N2CCC(NCC3NCCS3)CC2)CC1]

(non-covalent docking score, −9.10; covalent docking score, −4.249),

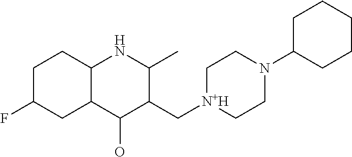

(non-covalent docking score, −9.28; covalent docking score, −4.614), and

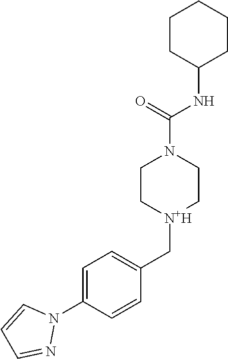

(non-covalent docking score, −9.321; covalent docking score, −4.874).

In some embodiments, the small molecule is a covalent compound. The covalent docking score, a measurement of binding in the Cys23 region, in some embodiments, is at least −4 (e.g., −4.1, −4.2, −4.3, −4.4, −4.5, −4.6, −4.7, −4.8, −4.9, −5.0, −5.25, −5.5, −5.75, −6.0, −6.1). Exemplary covalent small molecule inhibitors that target Cys23 of OTUB1 are as follows:

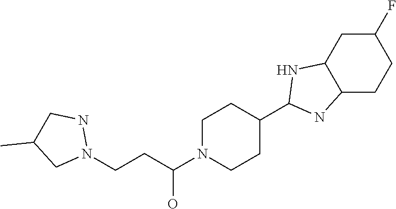 [SMILES: CC1CNN(CCC(O)N2CCC(C3NC4CCC(F)CC4N3)CC2)C1]

(covalent docking score, −5.579) and

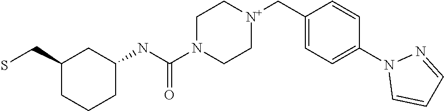 [SMILES: O=C(N[C@@H]1CCC[C@@H](CS)C1)N1CC[NH+](Cc2ccc(-n3cccn3)cc2)CC1]

(covalent docking score, −6.173).

In some embodiments, the agent is a glutaredoxin (GLRX). Mammalian glutaredoxins are members of the thiol-disulfide oxidoreductase family. They are often characterized by a thioredoxin fold and a Cys-Pro (Ser)-Tyr-Cys active site (SEQ ID NO: 16). Non-limiting examples include GRX1, a cytosolic protein; GRX2, which may be directed to the mitochondria by a mitochondrial leader sequence and/or can also occur in the nucleus following alternative splicing; and GRX5 a mitochondrial glutaredoxin, named GRX5 because it is homologous to yeast CRX5. GLRXs may deglutathionylating proteins, thereby reestablishing reduced protein thiol groups under physiological conditions. The enzymes can use glutathione as a cofactor, and may participate in certain cellular functions, such as redox signaling and the regulation of glucose metabolism. They can be oxidized by substrates, and reduced non-enzymatically by glutathione. In some embodiments, the GLRX comprises glutaredoxin-1 (Glrx). Glrx is 12 kDa protein, and is described, for example, in US 2014-0140975, the entire contents of which are incorporated herein in their entirety. In some embodiments, the GLRX comprises GRX1, GRX2, GRX5, or a modified version thereof. As used herein, “modified version” refers to a variant enzyme that has about the same activity as the wild-type version of the enzyme (for example, 70%, 75%, 80%, 85%, 90%, 91%, 92%, 93%, 94%, 95%, 96%, 97%, 98%, 99%, 100%, 101%, 102%, 103%, 104%, 105%, 106%, 107%, 108%, 109%, 110%, 115%<120%, 125%, 130%, or more enzymatic activity than the wild-type enzyme).

### Dosing/Administration

Provided herein in accordance with one set of embodiments are certain agents for the prevention and/or treatment of proliferative diseases (e.g., cancer) in humans and other mammals. “Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Hemopoietic cancers, such as leukemia, are able to outcompete the normal hemopoietic compartments in a subject, thereby leading to hemopoietic failure (in the form of anemia, thrombocytopenia and neutropenia) ultimately causing death. Many tumors have altered metabolic demand, and such altered antioxidant defenses may permit tumor growth.

Such effects may be countered, at least in part, by the application of the compositions discussed herein comprising a glutaredoxin, in accordance with certain embodiments. Examples of cancers that can be treated using the compositions of the disclosure include, but are not limited to: biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. In some embodiments, the cancer is one in which OTUB1 and/or SLC7A11 expression levels are elevated relative to a reference level (e.g., physiological levels, levels in non-cancerous tissues), for example, breast, ovarian, lung, liver, colon, glioma, melanoma, acute myeloid leukemia, esophageal, gastric, endometrial, prostate, and/or thyroid cancer. In some embodiments, the cancer is a lung cancer (e.g., non-small cell lung cancer, squamous cell carcinoma (epidermoid carcinoma), adenocarcinoma, large-cell carcinoma or small-cell lung cancer).

In some embodiments an effective amount of the agent is administered to a subject (e.g., a mammalian subject, such as a human or a non-human subject) to treat a cancer or multiple cancers. The agent may be administered by any route that results in a therapeutically effective outcome, including but not limited to intradermal, intramuscular, intranasal, and/or subcutaneous administration. An “effective amount” of an agent is based at least in part, on the tissue and/or cell type targeted, the means of administration, characteristics of the agent. Other determinants include the body weight, age, height, sex and general health of the subject. Typically, an effective amount of an agent treats a cancer. As used herein, “treat” to either therapeutic treatment or prophylactic or preventative measures, wherein the object is to prevent or lessen the targeted pathologic condition or disorder as described herein (e.g., cancer). Thus, in some embodiments, treating may include directly affecting or curing, suppressing, inhibiting, preventing, reducing the severity of, delaying the onset of, reducing symptoms associated with the disease, disorder or condition, or a combination thereof. In some embodiments, “treating” refers inter alia to delaying progression, expediting remission, inducing remission, augmenting remission, speeding recovery, increasing efficacy of or decreasing resistance to alternative therapeutics, or a combination thereof.

In some embodiments, administration of an agent provided herein, is used to treat a cancer, for example, by improving the killing of tumor cells, improving the subject's sensitivity to an immune checkpoint inhibitor (e.g., improving the efficacy of the immune checkpoint inhibitor therapy), increasing the subject's sensitivity to a chemotherapeutic, or any combination thereof.

Thus, in some embodiments, administration of an agent provided herein (e.g., an agent that inhibits or prevents the oxidation of OTUB1 Cys23 and/or Cys204) kills tumor cells. Examples of tumor cells which may be killed in a subject using the methods, compositions, and kits of the disclosure include, but are not limited to, central nervous system tumor cells, mesothelioma cells, lung cancer cells, non-small cell lung cancer cells, undifferentiated lung carcinoma cells, large cell lung carcinoma cells, adenocarcinoma cells, bronchoalveolar cell lung carcinoma cells, liver cancer cells, localized non-central nervous system tumor cells, solid tumor cells, and ovarian cancer cells. Improved killing of tumor cells means that administration of an agent described herein kills more tumor cells relative to a control in which the agent has not been administered.

In some embodiments, administration of an agent provided herein (e.g., an agent that inhibits or prevents the oxidation of OTUB1 Cys23 and/or Cys204) improves a subject's sensitivity to an immune checkpoint inhibitor (e.g., sensitivity to immune checkpoint inhibitor therapy). Therefore, in some embodiments, the agent is administered concomitantly with at least one immune checkpoint inhibitor. As used herein, “concomitant” refers to the administration of two or more materials/agents to a subject in a manner that is correlated in time, preferably sufficiently correlated in time so as to provide a modulation in a physiologic or immunologic response, and even more preferably the two or more materials/agents are administered in combination. In embodiments, concomitant administration may encompass administration of two or more materials/agents within a specified period of time, for example, within 1 month, 1 week, 1 day, 1 hour, or less. In certain embodiments, the two or more materials/agents are sequentially administered, and/or serially administered. In some embodiments, the materials/agents may be repeatedly administered concomitantly; that is concomitant administration on more than one occasion. Examples of immune checkpoint inhibitors include, but are not limited to CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligand inhibitors or a combination thereof.

As described herein, the administration of an agent (e.g., an agent that inhibits or prevents the oxidation of OTUB1 Cys23 and/or Cys204) can reduce chemotherapeutic resistance in certain cases. Thus, in some embodiments, the agent is administered concomitantly with an anti-cancer drug (e.g., chemotherapeutic). Examples of chemotherapeutic agents include, but are not limited to, methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MMI270, BAY 12-9566, RAS farnesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Placlitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT (Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Cactyx/liposomal doxorubicin, Gemzar/Gemcitabine, ZD 0473/Anormed, YM 116, iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide. Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′ deoxycoformycin), Semustine (methyl-CCNU), Teniposide (VM-26) and Vindesine sulfate. In some embodiments the chemotherapeutic agent comprises cisplatin.

### Pharmaceutical Compositions

Provided herein are pharmaceutical compositions comprising the agent with a carrier (e.g., a pharmaceutically acceptable carrier), inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences. The agent may be formulated or administered alone or in conjunction with one or more other components. For example, a pharmaceutical composition may further comprise other components, such as chemotherapeutics, immune checkpoint inhibitors, adjuvants, or combinations thereof.

Relative amounts of the agent, the pharmaceutically acceptable excipient, and/or any additional components in a pharmaceutical composition will differ. Pharmaceutical composition will depend upon the condition, age, weight, height or the subject, and additionally upon the route by which the composition will be administered. As an example, the pharmaceutical composition may comprise ≤100%, e.g., between 1 and 10%, between 5-50%, between 10-90%, at least 75% weight for weight (w/w) active ingredient. For the purposes of the present disclosure, the phrase “active ingredient” generally refers to the agents described herein.

Subject doses of the compounds described herein for mucosal or local delivery typically range from about 0.1 microgram to 10 mg per administration, which depending on the application could be given daily, weekly, or monthly and any other amount of time therebetween. More typically mucosal or local doses range from about 10 micrograms to 5 mg per administration, and most typically from about 100 micrograms to 1 mg, with 2 to 4 administrations being spaced days or weeks apart. More typically, doses range from 1 microgram to 10 mg per administration, and most typically 10 micrograms to 1 mg, with daily or weekly administrations. Subject doses of the compounds described herein for parenteral delivery for the purpose of treating asthma may be typically 5 to 10,000 times higher than the effective mucosal dose, and more typically 10 to 1,000 times higher, and most typically 20 to 100 times higher. More typically parenteral doses for these purposes range from about 10 micrograms to 5 mg per administration, and most typically from about 100 micrograms to 1 mg, with 2 to 4 administrations being spaced days or weeks apart. In some embodiments, however, parenteral doses for these purposes may be used in a range of 5 to 10,000 times higher than the typical doses described above. The compositions of the present disclosure may be administered in multiple doses over extended period of time. For any compound described herein the therapeutically effective amount can be initially determined from animal models. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

Administration of a composition of the disclosure may be accomplished by any medically acceptable method which allows the composition to reach its target. The particular mode selected will depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the composition within the subject without causing clinically unacceptable adverse effects.

The pharmaceutical composition may be formulated to be administered to the subject via any medically acceptable method. The administration may be localized (i.e., to a particular region, physiological system, tissue, organ, or cell type) or systemic, depending on the condition to be treated. For example, the composition may be formulated to be administered orally, vaginally, rectally, buccally, pulmonary, topically, nasally, transdermally, through parenteral injection or implantation, via surgical administration, or any other method of administration where access to the target by the composition of the disclosure is achieved. Examples of parenteral modalities that can be used with the disclosure include intravenous, intradermal, subcutaneous, intracavity, intramuscular, intraperitoneal, epidural, or intrathecal. Examples of implantation modalities include any implantable or injectable drug delivery system. Oral administration may be preferred in some embodiments because of the convenience to the subject as well as the dosing schedule. Compositions suitable for oral administration may be presented as discrete units such as hard or soft capsules, pills, cachettes, tablets, troches, or lozenges, each containing a predetermined amount of the active compound. Other oral compositions suitable for use with the disclosure include solutions or suspensions in aqueous or non-aqueous liquids such as a syrup, an elixir, or an emulsion. In some embodiments, the composition may be used to fortify a food or a beverage.

In some embodiments, the compositions of the disclosure are formulated for administered by inhalation. For administration by inhalation, the compositions for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein are formulations for pulmonary delivery of the pharmaceutical compositions of the present disclosure. The pharmaceutical compositions may be delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13 (suppl. 5): 143-146 (endothelin-1); Hubbard et al., 1989, Annals of Internal Medicine, Vol. III, pp. 206-212 (al-antitrypsin); Smith et al., 1989, J. Clin. Invest. 84:1145-1146 (a-1-proteinase); Oswein et al., 1990, “Aerosolization of Proteins,” Proceedings of Symposium on Respiratory Drug Delivery II, Keystone, Colo., March, (recombinant human growth hormone); Debs et al., 1988, J. Immunol., 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al. Contemplated for use in the practice of the methods described herein are a wide range of mechanical devices designed for pulmonary delivery of therapeutic products, including but not limited to nebulizers, metered dose inhalers, and powder inhalers, all of which are familiar to those skilled in the art. Non-limiting examples of commercially available devices suitable for the practice of this disclosure are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

Other delivery systems suitable for use with the present disclosure include time-release, delayed release, sustained release, or controlled release delivery systems. Such systems may avoid repeated administrations of the composition in many cases, increasing convenience to the subject. Many types of release delivery systems are available and known to those of ordinary skill in the art. They include, for example, polymer-based systems such as polylactic and/or polyglycolic acids, polyanhydrides, polycaprolactones and/or combinations of these; nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the composition. In addition, a pump-based hardware delivery system may be used to deliver one or more embodiments of the disclosure.

### Kits

The present disclosure also provides any of the above-mentioned compositions in kits, optionally including instructions for use of the composition for the treatment of a cancer. That is, the kit can include a description of use of the composition for participation in any biological or chemical mechanism disclosed herein. The kits can further include a description of activity of the condition in treating the pathology, as opposed to the symptoms of the condition. That is, the kit can include a description of use of the compositions as discussed herein. The kit also can include instructions for use of a combination of two or more compositions of the disclosure, or instruction for use of a combination of a composition of the disclosure and one or more other compounds indicated for treatment of the cancer. Instructions also may be provided for administering the composition by any suitable technique as previously described, for example, orally, intravenously, pump or implantable delivery device, or via another known route of drug delivery.

The kits described herein may also contain one or more containers, which may contain the composition and other ingredients as previously described. The kits also may contain instructions for mixing, diluting, and/or administrating the compositions of the disclosure in some cases. The kits also can include other containers with one or more solvents, surfactants, preservative and/or diluents (e.g., normal saline (0.9% NaCl), or 5% dextrose) as well as containers for mixing, diluting or administering the components in a sample or to a subject in need of such treatment.

The compositions of the kit may be provided as any suitable form, for example, as liquid solutions or as dried powders. When the composition provided is a dry powder, the composition may be reconstituted by the addition of a suitable solvent, which may also be provided. In embodiments where liquid forms of the composition are used, the liquid form may be concentrated or ready to use. The solvent will depend on the composition and the mode of use or administration. Suitable solvents for drug compositions are well known, for example as previously described, and are available in the literature. The solvent will depend on the composition and the mode of use or administration.

### EXAMPLES

In order that the present disclosure may be more fully understood, the following examples are set forth. The examples described in this application are offered to illustrate the compounds, pharmaceutical compositions, and methods provided herein and are not to be construed in any way as limiting their scope.

### Example 1. Glutathione is Increased in Response to Interleukin 1 Beta (IL1B) in a System xC- Dependent Manner and is Important in the Modulation of Inflammatory Cytokine Secretion

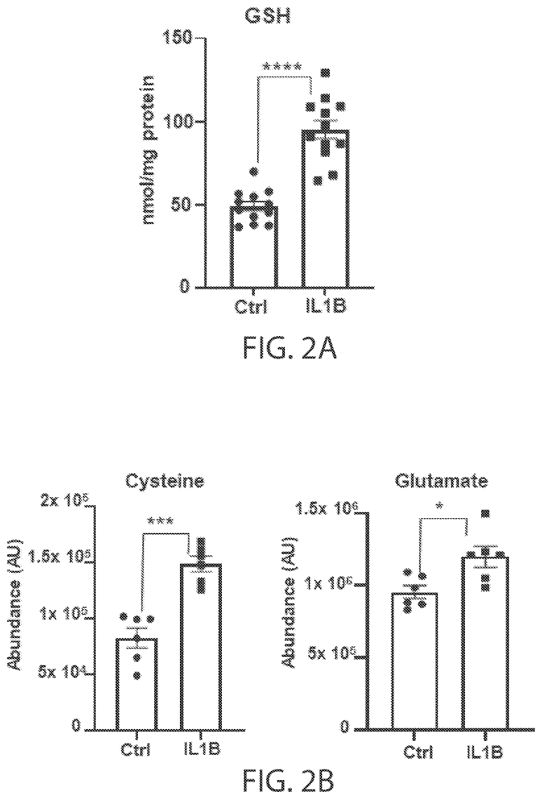

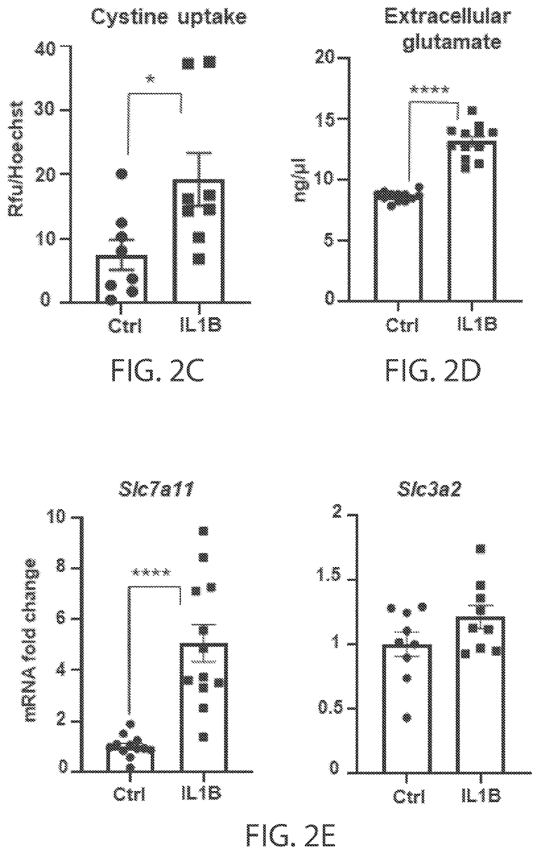

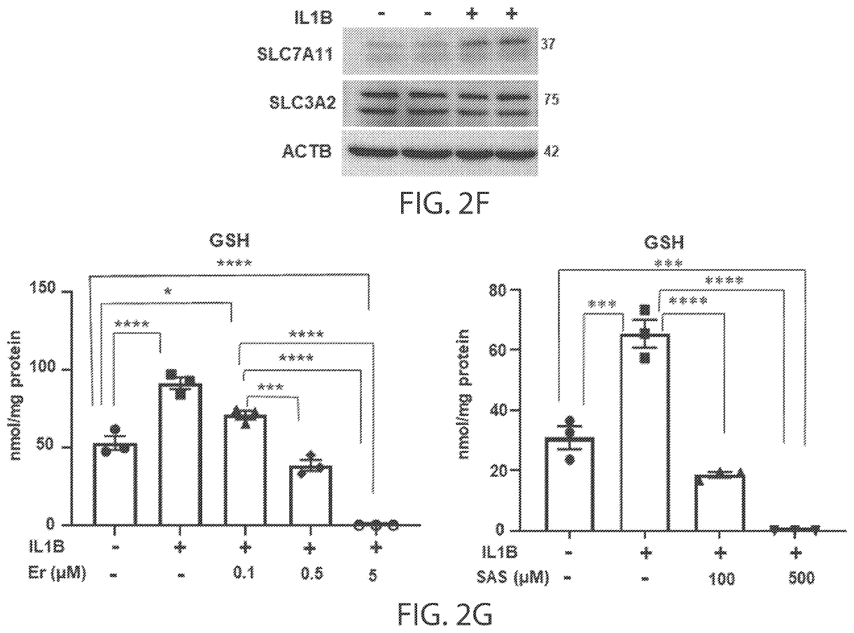

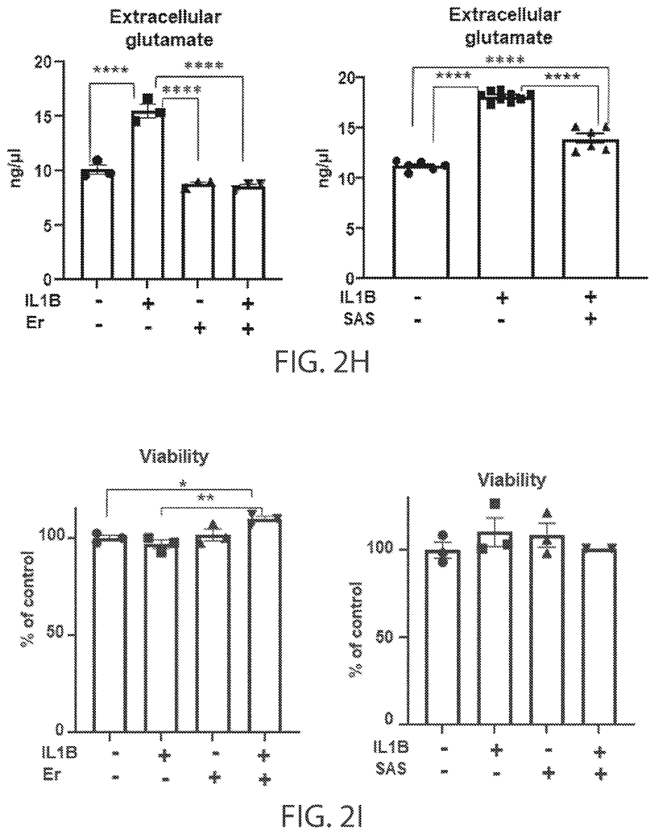

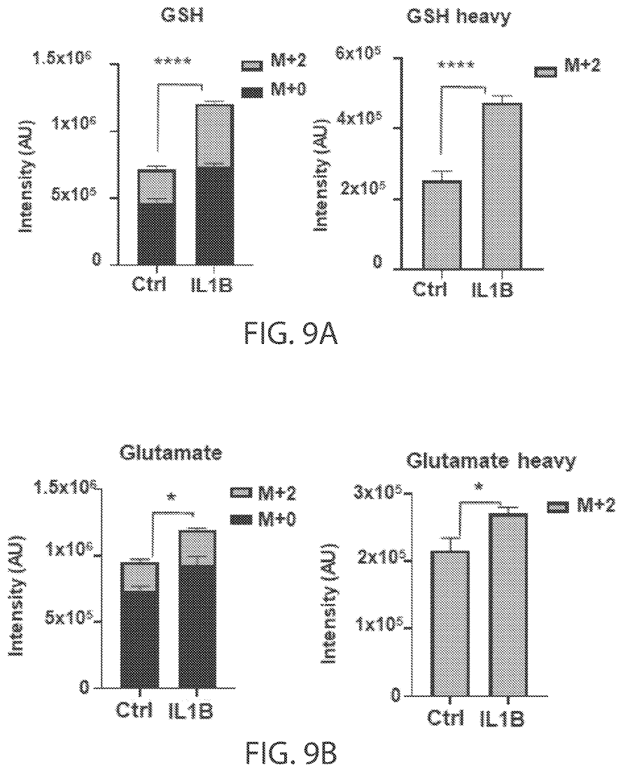

It has been previously shown that the pleotropic cytokine IL1B augments glycolysis in lung epithelial cells, and that interleukin 1B (IL1B) is linked to oxidative stress. The pentose phosphate pathway, an offshoot of glycolysis, regulates the redox environment by providing nicotinamide adenine dinucleotide phosphate (NADPH) reducing equivalents necessary for glutathione (GSH) homeostasis. The effects of GSH in response to IL1B were examined. Briefly, GSH was measured using a 5,5′-dithio-bis (2-nitrobenzoic acid) (DTNB) assay in both control and IL1B-stimulated mouse tracheal epithelial cells. Overall glutathione levels significantly increased in response to interleukin 1B (IL1B) (FIG. 2A). In addition, the levels of the unlabeled, labeled, and total GSH in control and IL1B stimulated cells for 24 hours were measured, and 13C-glucose tracing analysis demonstrated that carbons from glucose were used in synthesis of glutathione (GSH) (FIG. 9A). Statistics on the left panel represent the total GSH levels, without differentiating between labeled and unlabeled portions of GSH (P-value*<0.05, ****<0.0001).

Glutathione is a tripeptide composed of cysteine, glycine and glutamic acid. Levels of cysteine as well as glutamate were increased in response to IL1B, while glycine was not detected (FIG. 2B; intracellular cysteine, left; intracellular glutamate, right). In addition, levels of unlabeled, labeled and total glutamate in vehicle and IL1B treated epithelial cells were examined. Two of the glutamate carbons were also labeled (FIG. 9B), pointing to glutamate as the potential source of labeled carbons in GSH. Statistics on the left panel of FIG. 9B represent the total glutamate levels, without differentiating between labeled and unlabeled portions of glutamate (P-value*<0.05, ****<0.0001).

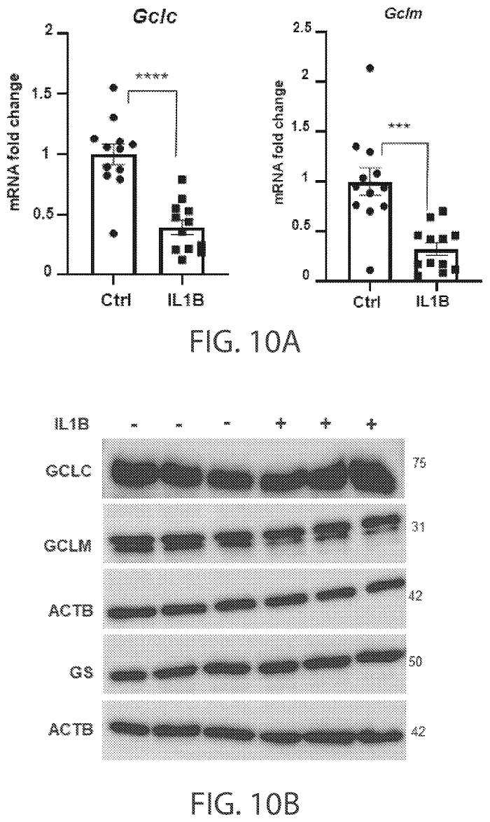

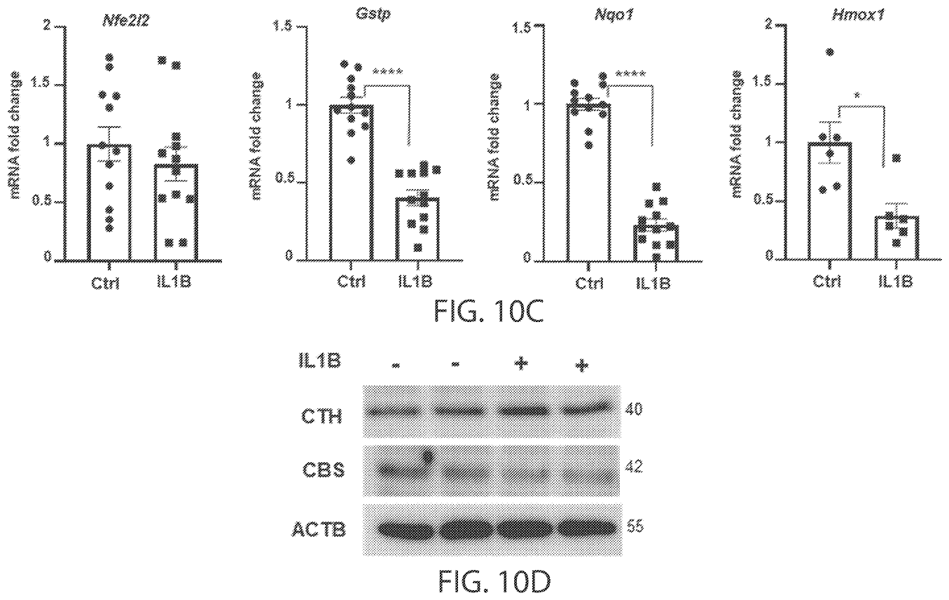

The mechanism(s) whereby IL1B increased GSH were next examined. Glutamate cysteine ligase (GCL), the rate limiting enzyme in GSH biosynthesis, is composed of the catalytic subunit GCLC and the modulatory subunit GCLM. mRNA expression levels of both GCLC and GCLM were significantly decreased by IL1B (FIG. 9A). A Western Blot analysis of GCLC, GCLM, GS, cystathionase (CTH) and cystathionine beta-synthase (CBS) proteins in response to 24 hours of IL1B stimulation was performed. ACTB was used as a loading control. Despite changes in mRNA, levels of GCLC, GCLM, did not change in response to IL1B, and glutathione synthase (GS) levels also remained unaltered (FIGS. 10B and 10D; numbers on the right refer to the protein molecular weight (MW) in kDa). In addition, mRNA levels of the transcription factor nuclear factor erythroid 2-related factor 2 (Nfe212), which is known to upregulate expression of multiple genes involved in antioxidant defense, did not significantly change in response to IL1B, while Glutathione S-transferase P (Gstp), NAD(P)H Quinone Dehydrogenase 1 (Nqo1) and Heme Oxygenase 1 (Hmox1), three downstream targets of nuclear factor erythroid 2-related factor 2 (NRF2), decreased after IL1B treatment (FIGS. 10A and 10C). Levels of cystathionase (CTH) and cystathionine β-synthase (CBS), members of the transsulfuration pathway as a potential source of cysteine also remained unchanged or decreased in response to IL1B (FIG. 10D). Collectively, these observations show that IL1B-induced increases in glutathione occur independent of changes in GCLC, GCLM, GS, CTH or CBS point to regulation of GSH independent from NRF2 or the transulfuration pathway.

A regulator of GSH homeostasis is the transporter system xC-, which exports glutamate and concomitantly imports cystine in a 1:1 ratio. Once inside the cells, cystine is reduced to two cysteines, the latter being the rate limiting amino acid required for GSH synthesis. These relationships were examined further. In addition to increases in intracellular cysteine (FIG. 2B), uptake of fluorescein isothiocyanate (FITC)-labeled cystine (FIG. 2C) and extracellular glutamate (FIG. 2D) in mouse tracheal epithelial cells were also significantly increased in response to IL1B. mRNA expression and protein levels of the system xC- subunit, solute carrier family 7 member 11 (SLC7A11), were also upregulated in response to IL1B, with no significant changes in solute carrier family 3 member 2 (SLC3A2) subunit (FIG. 2E, an mRNA analysis and FIG. 2F, and Western blot analysis with beta actin used as a loading control). To address whether IL1B-induced increases in GSH are system xC- dependent, two system xC- inhibitors, erastin (Er) and sulfasalazine (SAS) were used. Cells were pretreated for one hour, followed by IL1B treatment for 24 hours. Then, GSH levels were measured in cells exposed to increasing doses of Er or SAS (FIG. 2G). Extracellular glutamate levels in cell culture supernatant of cells pretreated with 0.5 micromolar Er (left) or 100 micromolar SAS (right) for 1 hour before IL1B stimulation were also measured (FIG. 2H). Both inhibitors significantly decreased IL1B-induced increases in GSH and extracellular glutamate levels (FIG. 2G-2H). System xC- inhibition is known to induce ferroptosis, an iron-dependent mode of cell death characterized by increased lipid peroxidation. However, no changes in cell viability using erastin (5 micromolar) or sulfasalazine (500 micromolar) were detected (FIG. 2I; P-value*<0.05, ***<0.001, ****<0.0001).

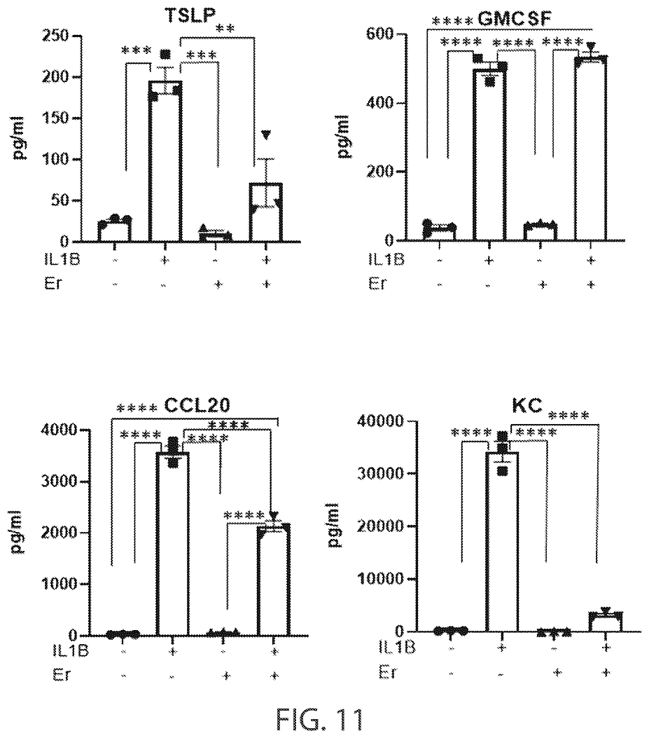

The secretion of various IL1B-induced pro-inflammatory cytokines were examined. Thymic stromal lymphopoietin (TSLP), granulocyte-macrophage colony-stimulating factor (GM-CSF), chemokine (C—C motif) ligand 20 (CCL20), and keratinocyte-derived chemokine (KC) cytokine levels secreted in cell culture supernatant of mouse tracheal epithelial (MTE) cells were evaluated after 1 hour pretreatment with erastin (0.5 micromolar) followed by 24 hours stimulation with IL1B (P value**<0.01, <0.001, ****<0.0001; FIG. 11). TSLP, CCL20, and KC were significantly attenuated by erastin (FIG. 11).

Collectively, these data demonstrate that system xC- is important in enhancing GSH levels in IL1B-stimutated cells and that GSH in turn promotes pro-inflammatory signaling independently of cell death.

### Example 2. IL1B Induces a Dithiotreitol (DTT)-Sensitive High Molecular Weight Complex Containing SLC7A11, SLC3A2 and Ovarian Tumor Deubiquitinase-1 (OTUB1)

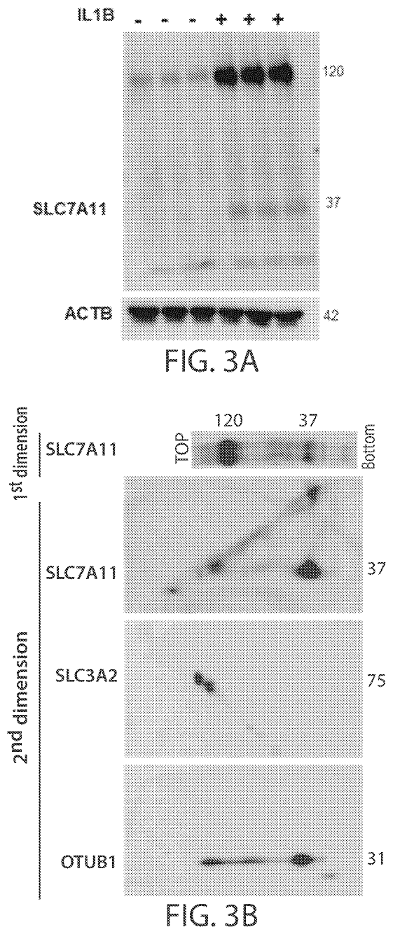

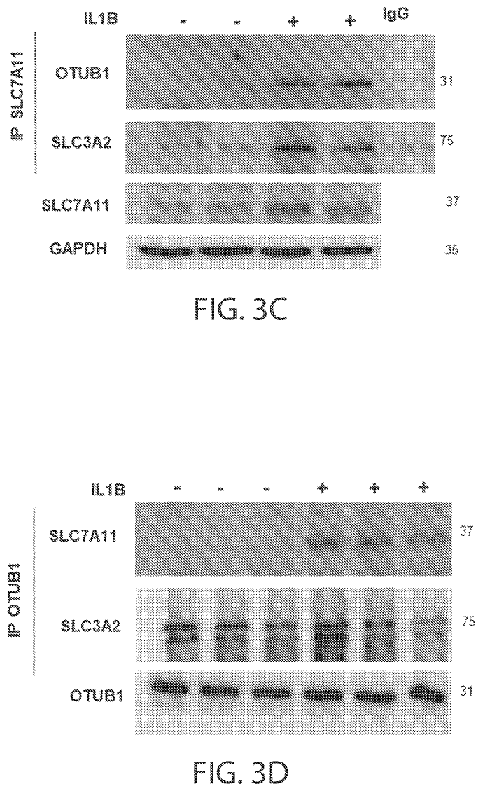

Ovarian tumor (OTU) deubiquitinase, OTUB1 is a target for protein S-glutathionylation (PSSG) in epithelial cells stimulated with IL1B. OTUB1, a member of the ovarian tumor proteases subfamily, directly interacts with and stabilizes SLC7A11 in cancer cells by preventing its ubiquitination. To assess whether system xC- was regulated in a redox-dependent manner, non-reducing Western Blots for SLC7A11 were performed, revealing the formation of a high molecular weight (HMW) species appearing at ˜120 kDa in MTE cells in the presence or absence of IL1B for 24 hours and that the HMW species increased in response to IL1B (FIG. 3A). 2D PAGE showed that SLC7A11. OTUB1, and SLC3A2 were detected in association with the ˜ 120 kDa complex (FIG. 3B) and co-immunoprecipitation analysis further confirmed the association between SLC7A11, SLC3A2 and OTUB1 after IL1B stimulation (FIG. 3C). Although the transmembrane glycoprotein CD44 is also known to bind and stabilize the SLC7A11 and OTUB1 complex, CD44 in lung epithelial cells in the absence or presence of IL1B stimulation was not detected (data not shown).

### Example 3. S-Glutathionylation Augments GSH and SLC7A11

Protein S-glutathionylation (PSSG) is a key regulator of biological responses induced by redox perturbations. PSSG occurs through addition of a glutathione moiety to a reactive cysteine in a target protein leading to changes in protein's structure and function.

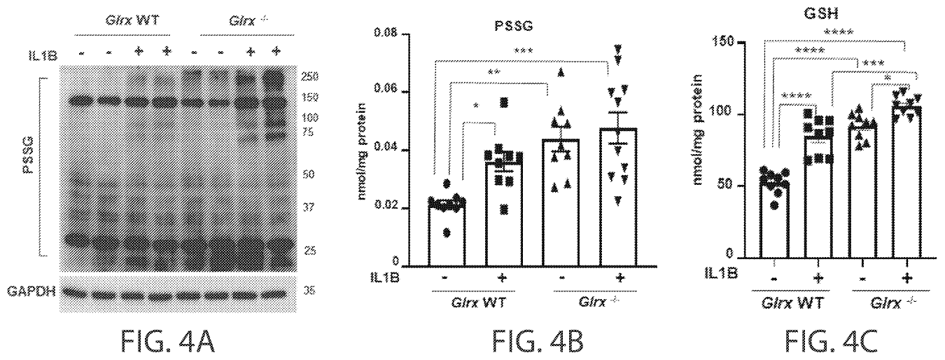

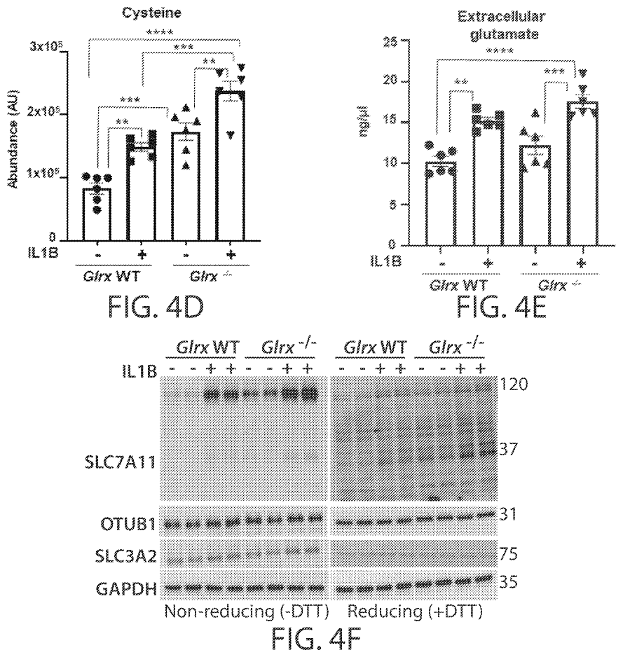

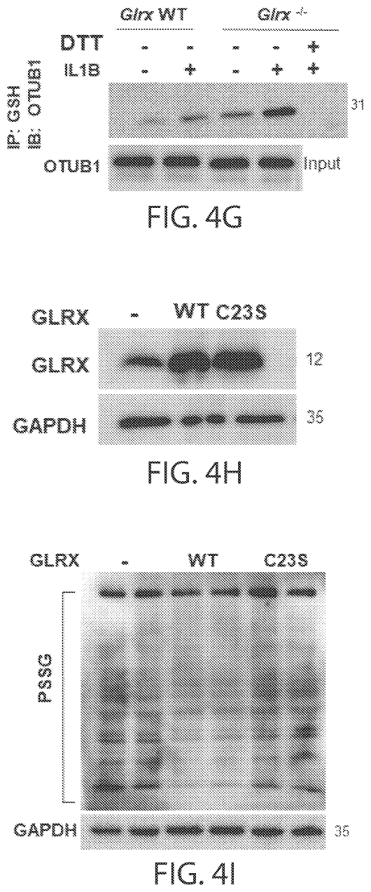

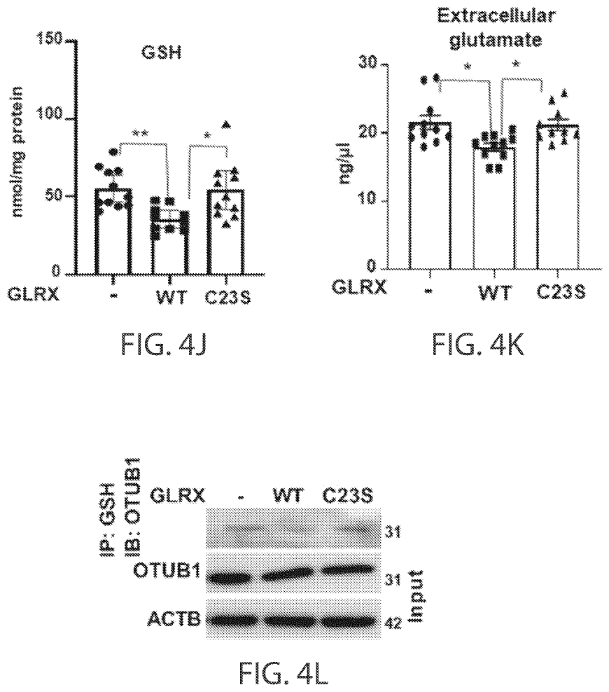

PSSG was assessed in wild-type (WT) and glutaredoxin-1 (Glrx)^{-/-} cells treated with vehicle or IL1B, with glyceraldehyde 3-phosphate dehydrogenase (GAPDH) used as a loading control. PSSG measurements were also taken using an DTNB assay in epithelial cells with or lacking Glrx and stimulated with IL1B for 24 hours. Overall PSSG was increased in response to IL1B (FIGS. 4A-4B). Glutaredoxin (GLRX) is the main enzyme is responsible for deglutathionylating proteins thereby re-establishing reduced protein thiol groups under physiological conditions. Cells lacking Glrx1 had further increases baseline or IL1B-stimulated PSSG (FIGS. 4A-4B). Total GSH levels were also significantly elevated in Glrx^{-/-} cells compared to wild-type (WT) cells and treatment with IL1B led to further increases in GSH in Glrx-MTE cells compared to the respective WT group (FIG. 4C). Intracellular cysteine was also increased in Glrx^{-/-} MTE cells compared to WT MTE cells after stimulation with vehicle or IL1B for 24 hours, while extracellular glutamate was not significantly different between WT and Glrx^{-/-} cells (FIGS. 4D-4E). Given the increases in GSH and cysteine in Glrx^{-/-} cells, GLRX effects on system xC- were assessed. Non-reducing (left) and reducing (right) Western Blots of SLC7A11, OTUB1 and SLC3A2 in WT and Glrx cells with GAPDH used as a loading control were performed. Under non-reducing conditions, Glrx^{-/-} cells exhibited higher levels of HMW SLC7A11 compared to control cells, with further elevations occurring following IL1B (FIG. 4F). Under reducing conditions, the SLC7A11 immunoreactivity was no longer observed as a prominent HMW complex, pointing to its oxidation-dependent nature. However, increases in the 37 kDa species of SLC7A11 were apparent in unstimulated Glrx^{-/-} cells or in response to IL1B, while levels of SLC3A2 and OTUB1 were not affected (FIG. 4F). These collective findings demonstrate that PSSG and GLRX regulate levels of and DTT-sensitive HMW complexes containing SLC7A11.

### Example 4. S-Glutathionylation Increases SLC7A11 Association with OTUB1

OTUB1 was one of the PSSG targets detected in IL1B-stimulated epithelial cells raising the possibility that S-glutathionylation of OTUB1 affects the stability of SLC7A11. To investigate this, immunoprecipitation of GSH followed by Western Blotting of OTUB1 in MTE cells treated with vehicle or IL1B was performed. Dithiothreitol (DTT) was incubated with cell lysates prior to immunoprecipitation as a negative control. OTUB1 levels from whole cell lysates were used as the input control. Increases in OTUB1 S-glutathionylation (OTUB1-SSG) in response to IL1B were observed, with further increases occurring in cells lacking Glrx (FIG. 4G), consistent with the role of GLRX as a deglutathionylating enzyme. Western blots were performed using cells treated with the WT- or C23S-mutant GLRX recombinant protein to examine the presence of glutaredoxin (FIG. 4H) and total PSSG (FIG. 4I) (P-value*<0.05, **<0.01, ***<0.001, ****<0.0001). GAPDH was used as a loading control. Glutathione was evaluated in MTE cells treated with recombinant WT- or C23S-mutant GLRX (FIG. 4J) and glutamate in cell culture supernatant of MTE cells treated with WT or the C23S mutant GLRX was also evaluated (FIG. 4K). Direct administration of recombinant WT GLRX protein to cells (FIG. 4H) decreased overall PSSG and GSH levels (FIGS. 4I-4J) and decreased extracellular glutamate (FIG. 4K), while catalytically inactive C23S GLRX did not elicit these effects. In a further experiment, immunoprecipitation of S-glutathionylated proteins was performed using a GSH antibody, followed by Western Blotting analysis of OTUB1 in cells treated with recombinant WT- or the C23S-GLRX recombinant protein. Total OTUB1 levels from whole cell lysates were used as the input control. It was found that wild-type GLRX, but not C23S GLRX, decreased OTUB1-SSG (FIG. 4L), pointing to a putative role of OTUB1-SSG in the regulation of GSH levels.

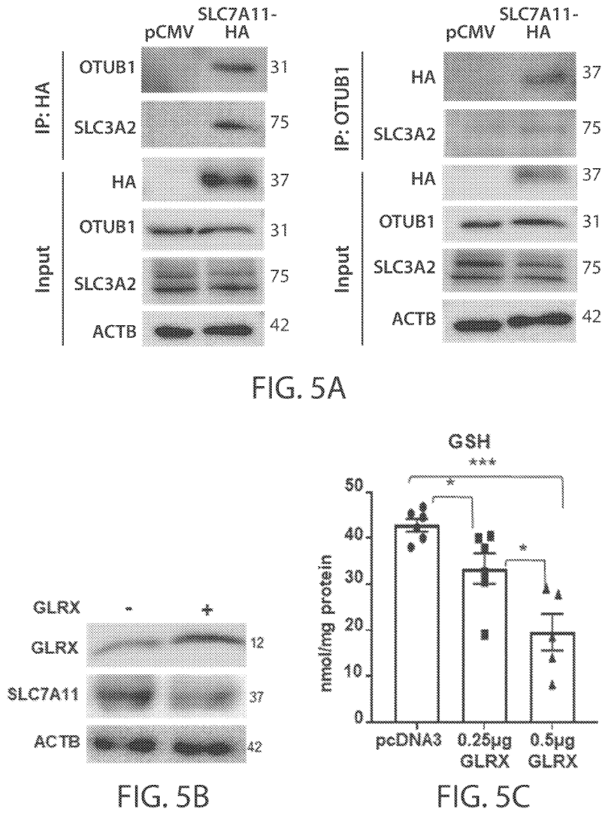

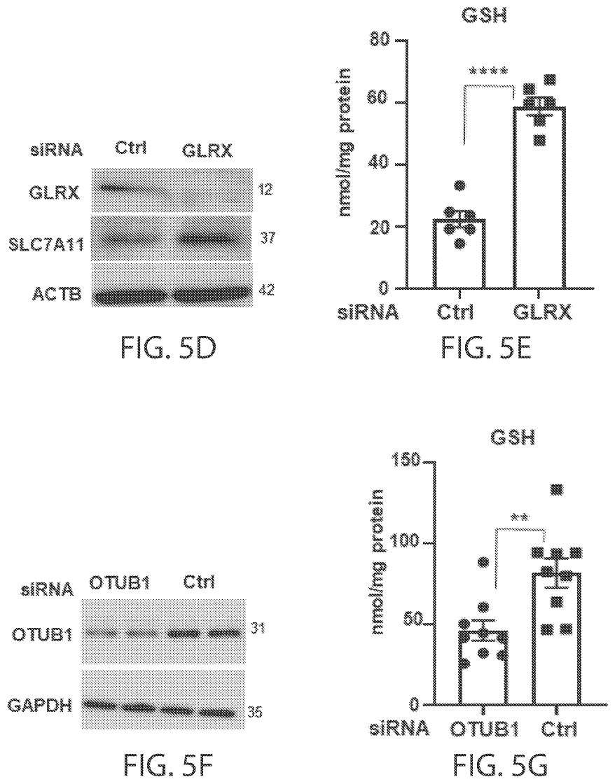

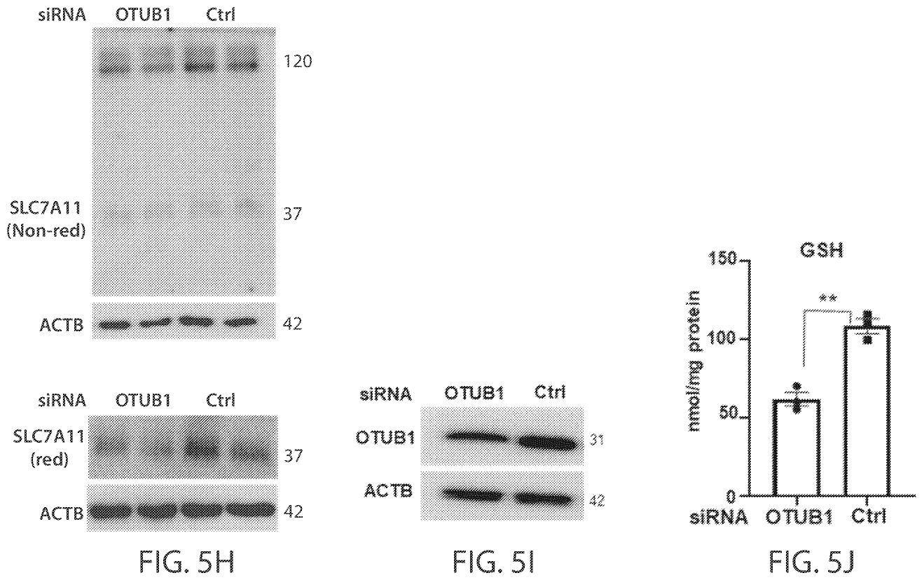

System xC- has been extensively studied in lung cancer where it controls sensitivity to ferroptosis. To unravel the functional importance of OTUB1-SSG in the regulation of GSH and to identify the specific S-glutathionylated cysteines in OTUB1, the H522 lung adenocarcinoma cell line was used. H522 cells express system xC- and are sensitive to GSH depletion. Immunoprecipitation of HA-tagged SLC7A11 or OTUB1 followed by Western blotting show constitutive interaction between HA-SLC7A11, SLC3A2, and OTUB1 (FIG. 5A). Beta actin (ACTB) was used as a loading control. Overexpression of GLRX in H522 cells decreased GSH (FIG. 5C) and SLC7A11 levels (FIG. 5B) (P-value*<0.05, **<0.01, ***<0.001). A pcDNA3 empty plasmid was used as a transfection control and ACTB was used as a loading control. Western blots of GLRX and SLC7A11 in H522 cells following GLRX siRNA-mediated knock down with ACTB used as a loading control. SiRNA-mediated knockdown of GLRX increased GSH and SLC7A11 levels (FIGS. 5D-5E) (P-value*<0.05, **<0.01, ***<0.001). SiRNA-mediated knock-down of OTUB1 diminished HMW SLC7A11 complex formation, overall levels of SLC7A11, and GSH in H522 cells (FIGS. 5F-5H). Knock-down of OTUB1 also decreased GSH in primary lung epithelial cells (MTE cells) (FIGS. 51-5J) (P-value*<0.05, **<0.01, ***<0.001). These findings collectively demonstrate functional importance of OTUB1 the regulation of system xC- and GSH levels.

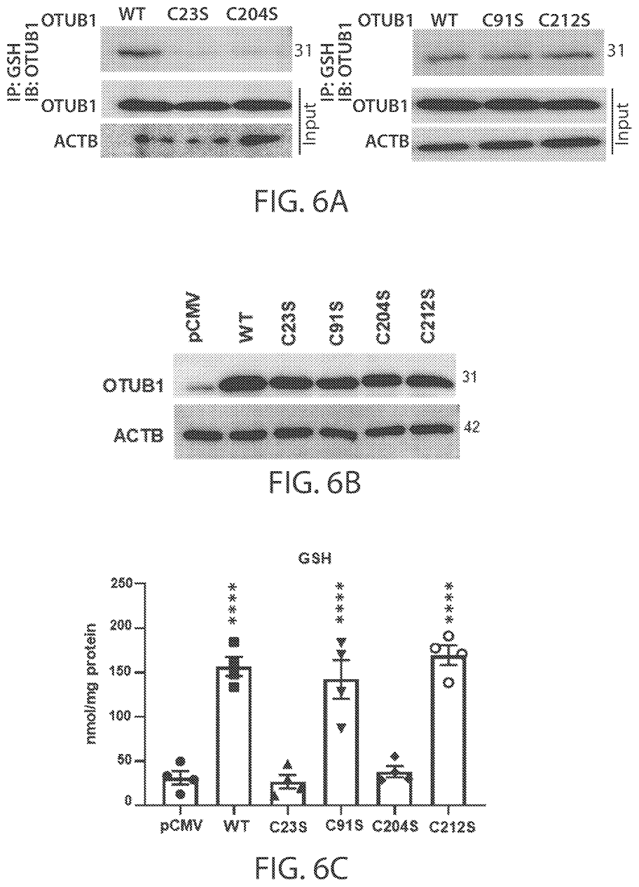

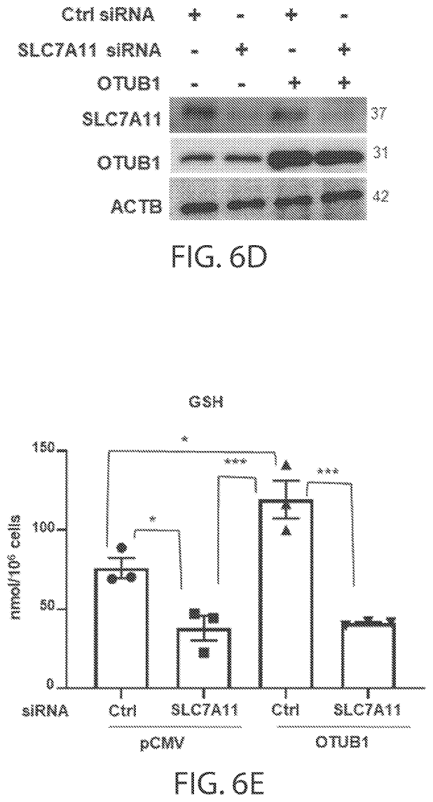

OTUB1 contains four cysteines (C23, C91, C204 and C212), with C91 required for OTUB1's canonical deubiquitinase activity. In order to identify the cysteines in OTUB1 that are targets for S-glutathionylation, each of the four cysteines were mutated to serines and expressed in H522 cells. The H522 cells overexpressed OTUB1-WT, OTUB1-C23S or OTUB1-C204S plasmids (FIG. 6A, left) and OTUB1-WT, OTUB1-C91S or OTUB1-C212S plasmids (FIG. 6A, right) and PSSG was immunoprecipitated using GSH antibody. OTUB1 and ACTB from total cell lysates were used as input. WT OTUB1, OTUB1-C91S and OTUB1C212S were constitutively S-glutathionylated. In contrast, OTUB1-C23S and OTUB1-C204S mutants were refractory to S-glutathionylation (FIG. 6A). A Western blot of OTUB1 in H522 cells transfected with pCMV, OTUB1 WT, OTUB1-C23S, OTUB1-C91S, OTUB1-C204S or OTUB1-C212S plasmids was performed, using ACTB as a loading control. While expression of WT, C91S and C212S mutants of OTUB1 increased GSH, the comparable expression of C23S- or C204S-OTUB1 mutants did not induce any significant increases in GSH (FIGS. 6B-6C) (P-value*<0.05, ***<0.001, **<0.01, ****<0.0001). These findings, that expression of C91S OTUB1 is sufficient to increase GSH levels indistinguishable from WT OTUB1, suggest a lack of canonical deubiquitinase activity in the regulation of GSH.

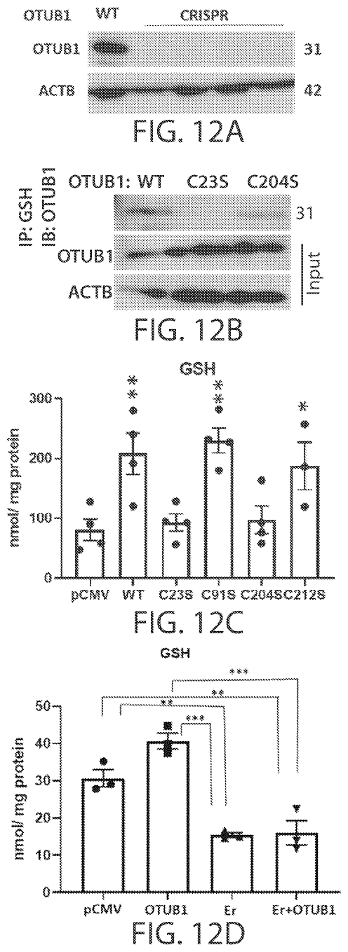

Similar results were obtained in H522 cells in which the OTUB1 gene was ablated via CRISPR-CAS9. GSH measurements from OTUB1-CRISPR H522 cells overexpressing OTUB1-WT, OTUB1-C23S, OTUB1-C91S, OTUB1-C204S or OTUB1-C212S plasmids were taken (FIG. 12A) and levels of OTUB1-SSG in OTUB1-CRISPR H522 cells overexpressing OTUB1-WT, OTUB1-C23S or OTUB1-C204S plasmids were measured (FIG. 12B). Western blots for OTUB1 and ACTB from whole cell lysates were used as input controls. GSH levels were also measured in H522 cells treated with 0.3 micromolar erastin and overexpressing OTUB1-WT plasmid (FIG. 12C). Re-expression of WT OTUB1, but not C23S or C204S mutants of OTUB1 elevated GSH (FIGS. 12A-12C).

In H522 cells transfected with control or SLC7A11 siRNA and overexpressed OTUB-WT or pCMV empty plasmid, with ACTB used as a loading control (FIG. 6D), the overexpression of WT OTUB1 did not induce increases in GSH when SLC7A11 was knocked down (FIG. 6E) (P-value*<0.05, ***<0.001, **<0.01, ****<0.0001). This demonstrates the requirement of SLC7A11 in the OTUB1-mediated increases in GSH. Overall, these results demonstrate that S-glutathionylation of OTUB1 at Cys23 or Cys204 augment GSH levels, independently of canonical deubiquitinase activity in association with stabilization of SLC7A11.

### Example 5. S-Glutathionylation of OTUB1 Promotes the Interaction of SLC7A11 And SLC3A2 and Protects System xC- from Ubiquitination

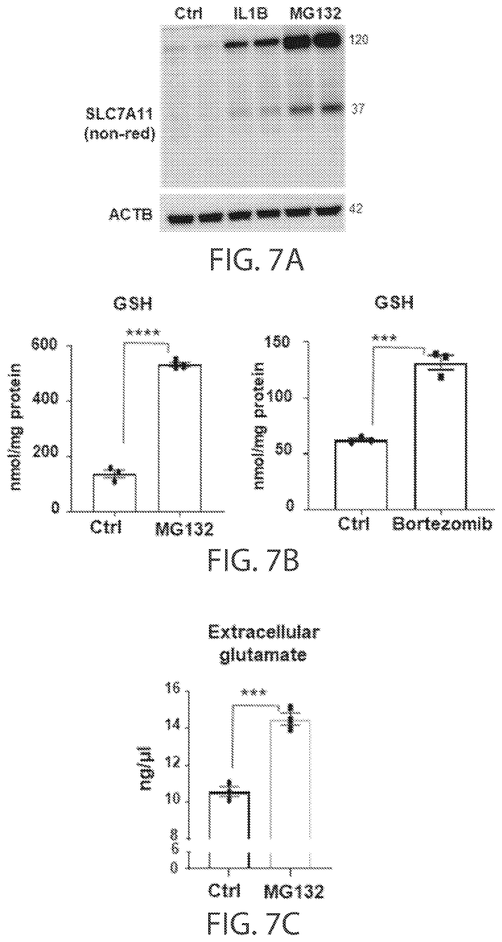

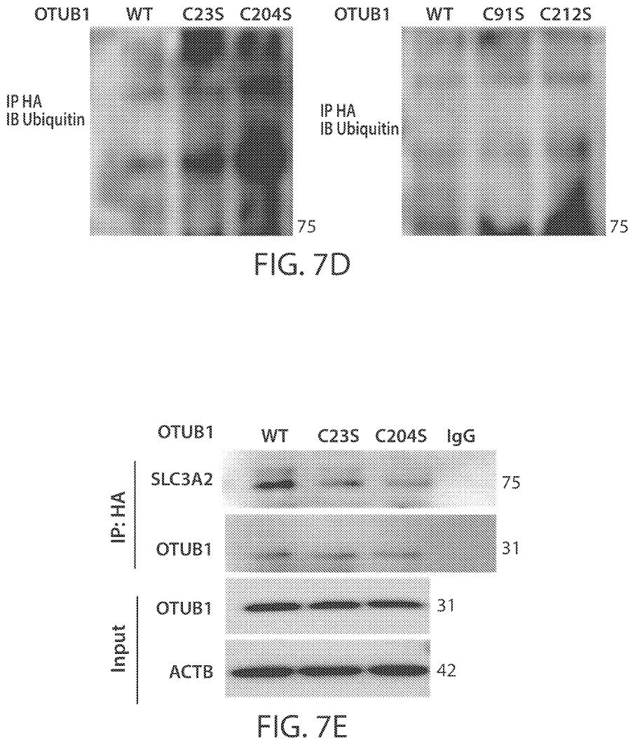

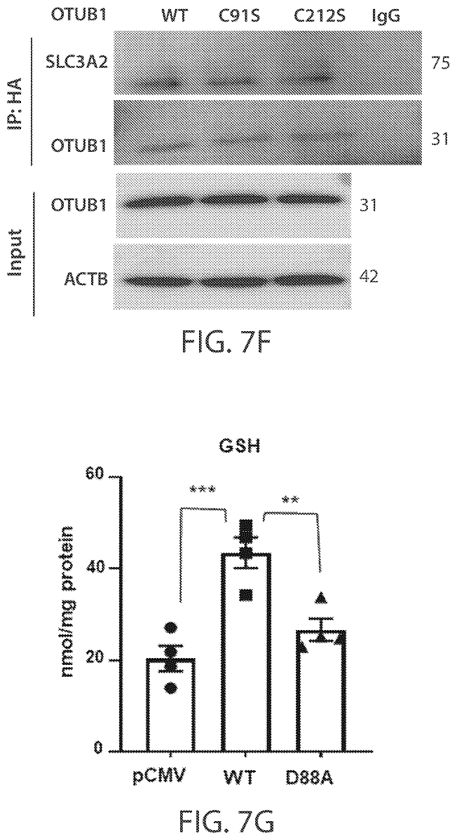

OTUB1 was previously shown to interfere with ubiquitination and subsequent proteasomal degradation of SLC7A11. A SLC7A11 non-reducing Western blot using MTE cells pretreated with 5 micrograms/ml MG132 or 10 ng/ml IL1B for 24 hours with ACTB used as a loading control was performed (FIG. 7A). In addition, an assessment of GSH levels in MG132-(5 micrograms/ml, 24 hours) (FIG. 7B, left) or bortezomib-(FIG. 7B, right) (0.3 micromolar, 24 hours) treated MTE cells was undertaken (P-value***<0.001, ****<0.0001). Extracellular glutamate levels in MG132-treated MTE cells were also assessed (5 micrograms/ml, 24 hours) (FIG. 7C) (P-value***<0.001, ****<0.0001). The proteasome inhibitors MG132 or bortezomib increased SCL7A11, GSH and extracellular glutamate levels in tracheal epithelial cells (FIGS. 7A-7C), identifying the importance of proteasomal degradation of SLC7A11 in regulating system xC- and glutathione levels.

Expression of WT OTUB1, OTUB1-C91S or OTUB1-C212S decreased ubiquitination of SLC7A11, while OTUB1-C23S or OTUB1-C204S did not affect ubiquitination of SLC7A11 (FIG. 7D). Additionally, both OTUB1-C23S and OTUB1-C204S mutants overexpression decreased the interaction between SLC7A11 and SLC3A2 suggesting a less functional system xC- (FIG. 7E), as shown by immunoprecipitation of HA in H522 cells overexpressing SLC7A11-HA tagged and OTUB1-WT. OTUB1-C23S or OTUB1-C204S followed by blotting for SLC3A2 and OTUB1 (OTUB1 and ACTB were used as input controls). OTUB1-C91S as well as OTUB1-C212S mutants' overexpression however promoted similar interaction between SLC7A11 and SLC3A2 compared to OTUB1-WT (FIG. 7F), as shown by immunoprecipitation of HA in H522 cells overexpressing SLC7A11-HA tagged and OTUB1-WT, OTUB1-C91S or OTUB1-C212S followed by blotting for SLC3A2 and OTUB1 (OTUB1 and ACTB were used as input controls).

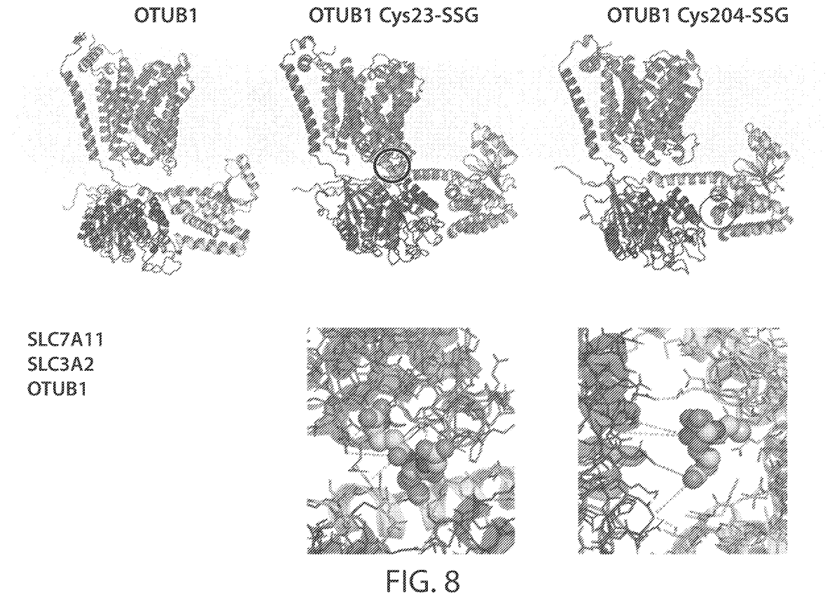

OTUB1 can inhibit ubiquitination of multiple proteins in a non-canonical manner, in which it directly interacts with and inhibits E2 conjugating enzymes. OTUB1 Aspartate 88 has been shown to bind the E2 conjugating enzymes and to suppress Ub-conjugating activity. To address whether non-canonical activity of OTUB1 is important in increasing GSH, a D88A mutant of OTUB1 was expressed. Results in FIG. 7G show that expression of D88A OTUB1 failed to induce increases in GSH levels in H522 cells, confirming the non-canonical function of OTUB1 in augmenting GSH. Molecular simulations wherein the impact of S-glutathionylation of OTUB1-C23S or OTUB1-C204S for the organization of the tri-protein complex was assessed. Without any PSSG, the tri-protein complex drifted away from the initial structure. Notably, the region of SLC3A2 that is not in the membrane moved further away and formed no contact with the bottom of SLC7A11. In contrast, S-glutathionylation of OTUB1 at either the C23 or the C204 position resulted in apparent stabilization of the complex with interactions between SLC7A11 and SLC3A2 being enhanced (FIG. 8).

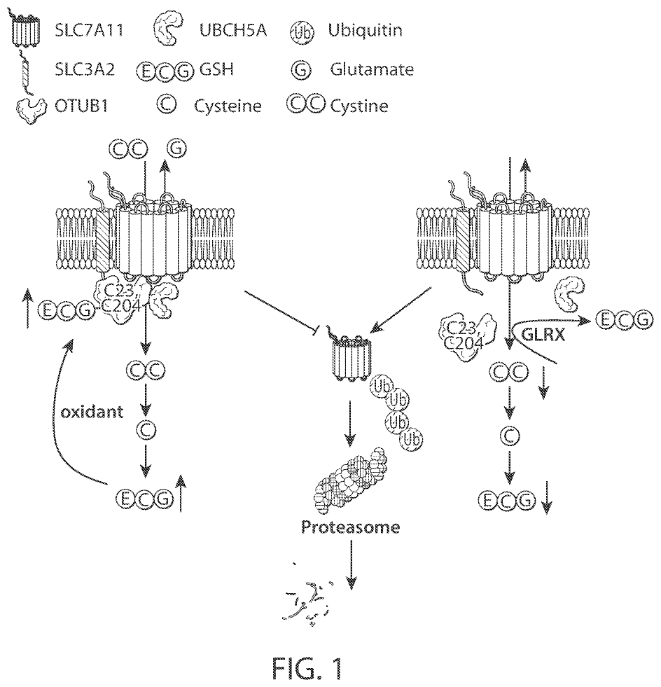

These observations highlight a mechanism of regulation of system xC- through glutathione-dependent protein oxidation that is under glutaredoxin-mediated control (FIG. 1). That is, OTUB1 may interact with and stabilizes solute carrier family 7 member 11 (SLC7A11), the active subunit of system xC- transporter. An oxidant signal may lead to increases in OTUB1 S-glutathionylation (OTUB1-SSG), specifically at Cys23 and Cys 204, which in turn stabilize SLC7A11, decreasing its ubiquitination and degradation. The resulting increase in SLC7A11 may allow cystine uptake, which can be incorporated in glutathione (GSH) synthesis following its reduction to cysteine intracellularly.

### Example 6. GLRX Increases Cisplatin-Induced Killing in Association with Decreasing GSH

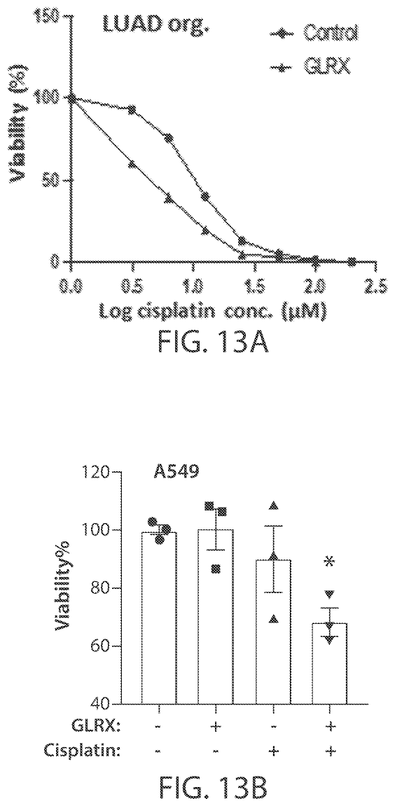

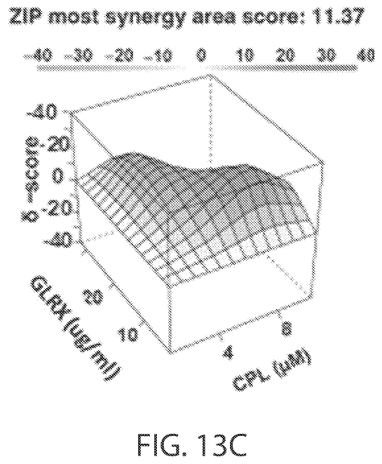

To date, the first line therapy for advanced KRAS-mutant lung adenocarcinoma (LUAD) consists of platinum-based chemotherapy in combination with the immune checkpoint inhibitor, pembrolizumab, or pembrolizumab alone in patients whose tumors have PD-L1 expression equal or greater than 50%. Cisplatin has been shown to augment anti-tumor immunity. However, tumor resistance to platinum and toxicity is limited in its effectiveness, necessitating the development of strategies to increase sensitivity to cisplatin-induced killing and increase responsiveness to immune checkpoint inhibitors. Increases in GSH contribute to resistance of cisplatin-induced killing. In one study we administered 20 μg/ml of GLRX to human LUAD organoids in the presence or absence of cisplatin. While GLRX itself did not affect viability, GLRX strongly increased the sensitivity to cisplatin-induced tumor organoid killing, compared to tumors only treated with cisplatin (FIG. 13A). Similarly, in KRAS-mutant A549 cells, GLRX promoted cisplatin-induced killing, while administration of GLRX alone did not decrease viability (FIG. 13B). Assessment of synergy using the Chou-Talalay method (Synergy Finder) revealed significant synergy (ZIP Score >10) between GLRX and cisplatin in killing of H522 cells (FIG. 13C).

Administration of GLRX to H522 LUAD cells or overexpression of GLRX in H522 LUAD cells also decreased SLC7A11 and GSH levels (FIGS. 5B-5C), pointing to the GLRX-dependent control of GSH levels via the OTUB1-system xC- axis in the control of cisplatin resistance.

### Example 7. Discussion

The glutaredoxin-S-glutathionylation redox axis has emerged as a key regulator of cellular processes as it controls protein structure and function and prevents overoxidation of reactive cysteines within proteins. Numerous S-glutathionylation targets in diverse pathways have been described and with advances in redox proteomics, the list of glutathionylated protein targets continues to grow. To date, the interplay between GLRX and glutathione levels has largely remained unknown. In the present study, a new dimension of GLRX's action is described through a demonstration that GLRX controls glutathione levels via the regulation of system xC-. In particular, it is demonstrated that S-glutathionylation of OTUB1 at Cys23 or Cys204 regulates SLC7A11 stability through a non-canonical mechanism that enhances the interaction between SLC7A11 and SLC3A2, stabilizing the System xC- complex, in turn leading to cystine uptake and increases in GSH.

OTUB1, a member of the ovarian tumor proteases subfamily, was recently identified as a key regulator of SLC7A11. It was demonstrated that CD44 promoted the interaction between OTUB1 and SCL7A11 and that the CD44-mediated enhancement of SLC7A11 stability depended on OTUB1. However, a CD44 interaction with SLC7A11 in primary lung epithelial cells or in LUAD H522 cells was not detected in the present work. OTUB1 has been previously shown to inhibit ubiquitination of multiple proteins in a non-canonical manner, through the interaction with and inhibition of E2 conjugating enzymes. Stabilization of SLC7A11 by OTUB1 has been speculated to involve directly binding between OTUB1 and SLC7A11 and the inhibition of E2-conjugating enzymes. The demonstration herein that OTUB1-C91S increases SLC7A11 and GSH similar to WT OTUB1, and that OTUB1-D88A disrupts increases in GSH confirm a non-canonical mechanism of action of OTUB1 in the stabilization of SLC7A11. Of note, OTUB1 recognizes both ubiquitin-charged E2 and free ubiquitin, and together the E2-linked and free ubiquitin mimic the configuration of a cleaved K48-linked di-ubiquitin, the product of canonical deubiquitinase activity. Intriguingly, the contact regions of the E2 Ubiquitin ligase, UbcH5b, and free ubiquitin with OTUB1 involve amino acids in proximity to Cys204 as well as Cys23. Whether the presence of a glutathione moiety at either cysteine affects E2 conjugating enzyme or free ubiquitin binding to OTUB1 requires further study. In addition to SLC7A11, OTUB1 also has other targets including multiple oncoproteins such as forkhead box protein M1 (FOXM1), tumor protein P53, cellular inhibitor or apoptosis protein (cIAP) and murine double minute X (Mdmx) which it stabilizes through a non-conventional mechanism. Further studies are necessary to address whether enzymes S-glutathionylation of OTUB1 also regulates those targets

The OTUB1-mediated stabilization of SLC7A11 following its S-glutathionylation depicts a regulatory feed forward mechanism whereby glutathione, in a form of a protein-mixed disulfide with OTUB1, regulates its own synthesis in an SLC7A11-dependent manner. Besides the system xC- mediated import of cystine, the trans-sulfuration pathway also provides a source of cysteine. As described above, CBS catalyzes the first and rate-limiting step in the trans-sulfuration pathway to convert homocysteine to cysteine. S-glutathionylation of Cys346 has been reported to augment the activity of CBS, leading to increases in cysteine, thus providing another regulatory mechanism, besides OTUB1-SSG, whereby increases in S-glutathionylation lead to increases in glutathione.

The importance of system xC- and GSH levels in IL1B-induced pro-inflammatory signaling in primary airway epithelial cells, independently of ferroptosis, was also studied. The results showing diminished secretion of proinflammatory cytokines, notably TSLP in IL1B-stimulated epithelial cells following GSH inhibition by erastin, point to the importance of GSH and system xC- in regulating pro-inflammatory signaling. Previous studies showed that absence of Glrx enhanced glycolysis and promoted IL1B-stimulated TSLP. IL1B-stimulated glycolysis in epithelial cells primes epithelial cells for subsequent stimulation with allergens and contributes to allergic airway disease. Thus, analogous to the glucose-dependence of SLC7A11-expressing tumors, primary airway epithelial cells stimulated with IL1B also rely on glucose and SLC7A11 expression to induce pro-inflammatory cytokines.

As it is described herein, OTUB1 S-glutathionylation stabilizes SLC7A11, increases the SLC7A11 and SLC3A2 interaction, enhancing system xC- activity, and subsequently glutathione levels have important implications for tumor biology. Enhanced expression of OTUB1 and SLC7A11 occur in numerous cancers. Increased OTUB1 expression is associated with aggressive disease, poor prognosis, and worse patients survival. SLC7A11 expression is positively correlated with chemotherapeutic resistance and worsening survival in cancer patients. Cystine uptake requires reduction to cysteine, a process that consumes NADPH. SLC7A11-expressing tumors were found to be reliant on glucose metabolism and activation of the pentose phosphate pathway in order to maintain redox control. Limitation of the glucose supply or inhibition of glucose transporters resulted in selective killing of SLC7A11-high cancer cells and suppressed tumor growth. The current findings, that link GLRX status to OTUB1 S-glutathionylation and SLC7A11 expression warrant further investigation into the status of OTUB1 S-glutathionylation and GLRX activity in SLC7A11-expressing tumors.

Overall, results from the present studies provide new insights into the regulation of system xC- by illuminating its control by GLRX and S-glutathionylation of OTUB1. These findings demonstrate a positive regulatory mechanism whereby S-glutathionylation augments GSH synthesis and have potential impact for the management of diseases that are accompanied by increases in activity of system xC-.

### Example 8. Materials and Methods

### Cell Culture

Primary mouse tracheal epithelial (MTE) cells were isolated from C57BL/6NJ wild-type or C57BL6/NJ mice lacking Glrx1 gene and cultured. Cells were grown on collagen coated plastic plates or transwell inserts with media change every 2 days until confluency. For interleukin 1B (IL1B) treatment: cells were incubated in plain Dulbecco's Modified Eagle Medium (DMEM): F12 media containing 6 mM glucose and 2 mM glutamine overnight followed by stimulation with 10 ng/ml IL1B or 0.1% bovine serum albumin (BSA) in phosphate buffered saline (PBS) (vehicle control) for 24 hours. Treatment with crastin, sulfasalazine or BSO occurred 1 hour before IL1B stimulation. Media and cells were collected for further analysis. For growing H522 and OTUB1-CRISPR-H522 cells: Roswell Park Memorial Institute (RPMI) media supplemented with 10% fetal bovine serum (FBS) and 1% pen/strep was used to grow the cells. Plasmid over-expression and OTUB1 knock-down experiments were done when cells were 60-70% confluent.

### ¹³C-Glucose Labeling and Metabolomics Analysis

MTE cells were grown to confluency in 6 well transwells as described above. After reaching confluency, cells were incubated overnight in plain DMEM:F12 media containing 6 mM glucose and 2 mM glutamine. The next day, cells were washed thoroughly in glucose free media before stimulating with IL1B or 0.1% BSA in PBS in media containing 6 mM of 13C-glucose and 2 mM glutamine. 24 hours after, cells were pelleted and mass spectrometry-based metabolomics was performed at University of Colorado, School of Medicine Metabolomics Core.

### Western Blotting and 2D Gel Analysis

Bio-Rad DC protein estimation kit was used to determine protein concentration in cell lysates. Equal amounts of proteins were resolved using sodium dodecyl sulfate (SDS)-polyacrylamide gel electrophoresis and transferred to polyvinylidene difluoride (PVDF) membranes, blocked in 5% BSA and incubated with primary antibodies overnight. Membranes were subsequently incubated with peroxidase-conjugated secondary antibodies for one hour and then visualized using chemiluminescence.

For 2D gels: The first-dimension lysates were run in the same manner as described above with omitting dithiothreitol (DTT) from the lysis buffer. Gel strips were cut and incubated in Nupage sample buffer containing 50 mM DTT for 15 minutes at room temperature. After decanting the solution, gel strips were placed in alkylating solution containing 50 mM dimethylamine (DMA) for 15 minutes. Lastly, the strips were quenched in a solution containing 5 mM DTT and 20% ethanol before running in the second dimensional SDS-PAGE.

For immunoprecipitation assays: 150-250 micrograms of proteins were used in the pull-down assays. One hour incubation with 50 mM DTT was used as a negative control for S-glutathionylated immunoprecipitated protein.

### Total GSH and PSSG Measurement

Cell lysates were prepared in 100 mM potassium phosphate buffer containing 0.6% sulfosalicylic acid, 1 mM ethylenediaminetetraacetic acid (EDTA) and 0.01% triton-x-100. To measure total glutathione (GSH), equal amounts of proteins were incubated with 3 micromolar 5,5′-dithio-bis (2-nitrobenzoic acid) (DTNB) reagent and 2.25 microgram/ml glutathione reductase followed by the addition of 240 micromolar Nicotinamide adenine dinucleotide phosphate (NADPH). Kinetic absorbance was measured every minute at 412 nm for 20 minutes. For protein S-glutathionylation (PSSG) measurement, 400 micrograms of protein was acetone precipitated and washed thoroughly to remove any free GSH. Pellets were reconstituted in 100 mM potassium phosphate buffer with 1 mM EDTA. Samples were then incubated with 1 mM sodium borohydride (NaBH4) or water (negative control) for 1 hour at room temperature. 10% metaphosphoric acid was added to all the samples, including controls, and left on ice for 10 minutes. Samples were centrifuged at 1000×g for 15 minutes and 20 microliters were loaded in a 96 well plate to measure PSSG using, 5′-dithio-bis-(2-nitrobenzoic acid) (DTNB) recycling enzyme method as stated above.

### Glutamate Measurement

Glutamate in media was measured using Biovision kit following manufacturer's protocol.

### Calcein AM Viability

Invitrogen Live/Dead viability/cytotoxicity kit was used to measure cell viability according to manufacturer's instruction. Data was expressed as percentage survival compared to control untreated cells.

### Real-Time Quantitative PCR (Q-PCR)

RNeasy mini columns were used to extract total RNA according to manufacturer's instruction. First strand c-DNA was synthesized using 0.5 microgram RNA and used for reverse transcribed gene analysis using SYBR Green. cDNA was further amplified by real-time quantitative PCR with Gc1c, Gc1m, Nfe212, Gstp, Nqo1, Hmox1, Slc7a11 and Slc3a2 primers. Data was normalized to Actb.

### Enzyme-Linked Immunosorbent Assay (ELISA)

TSLP, GM-CSF, CCL20 and KC levels in cell culture supernatants were detected using enzyme-linked immunosorbent assay (ELISA) kits according to manufacturer's protocol.

### Molecular Modeling:

Molecular simulations were conducted by the incorporation of the tri-protein complex, originally created by protein-protein docking. The OTUB1 model was docked into the SLC7A11-SLC3A2 complex model (from PDB: 7CCS). These complex models (three proteins+membrane) were simulated in water for 100 ns and the final snapshots were displayed.

### CRISPR/CAS9 Generation

CRISPR/CAS9-mediated knockout of OTUB1 in H522 cells were generated as previously described (63, 64). Briefly, three 20 bp targeting OTUB1 sequences were generated (sgRNA1 forward: CAC CGG GAT GTA CGA GTA CTT TTT G (SEQ ID NO: 1), sgRNA1 reverse: AAA CCA AAA AGT ACT CGT ACA TCC C (SEQ ID NO: 2), sgRNA2 forward: CAC CGA TCC GCA AGA CCA GGC CTG A (SEQ ID NO: 3), sgRNA2 reverse: AAA CTC AGG CCT GGT CTT GCG GAT C (SEQ ID NO: 4), sgRNA3 forward: CAC CGA GGC CAG ACA GTT AAC ACC T (SEQ ID NO: 5), and sgRNA3 reverse: AAA CAG GTG TTA ACT GTC TGG CCT C (SEQ ID NO: 6)) and transfected in H522 cells followed by flow sorting for positively transfected cells. Only cells that successfully exhibited OTUB1 knockout were propagated and used in experiments.

### Plasmids and Mutagenesis

Human untagged clone of OTUB1 was purchased from Origene (Catalogue number #SC108722) and site directed mutagenesis primers were designed using Agilent QuickChange primer design. For C23S: 5′-catcataggccagactgttaacaccttcggagtcg-3′ (SEQ ID NO: 7) and 5′-cgactccgaaggtgttaacagtctggcctatgatg-3′ (SEQ ID NO: 8), for C91S: 5′-agcccgatagaaactgttgccgtcaggcc-3′ (SEQ ID NO: 9) and 5′-ggcctgacggcaacagtttctatcgggct-3′ (SEQ ID NO: 10), for C204S: 5′-cacctcctgctggctgaactccttgacag-3′ (SEQ ID NO: 11) and 5′-ctgtcaaggagttcagccagcaggaggtg-3′ (SEQ ID NO: 12) for C212S: 5′-tcgctctccttgctcatgggctccacc-3′ (SEQ ID NO: 13) and 5′-ggtggagcccatgagcaaggagagcga-3′ (SEQ ID NO: 14) were used.

### Statistical Analysis

Experiments described herein were repeated at least 3 times with n=3 per group (unless otherwise stated). Results are expressed as means+/−SEM. Analysis and statistical differences were determined using GraphPad Prism software (Graphpad 8.2.1) using t-test (for groups of two) or one-way ANOVA with Tukey post hoc correction test for multiple comparisons. * indicates P value<0.05, ** indicates P value<0.01, *** indicates P value<0.001, **** indicates p value<0.0001.

### Example 9. S-Glutathionylation of OTUB1 Enhances Interaction with the E2 Ubiquitin Conjugating Enzyme, UBCH5A

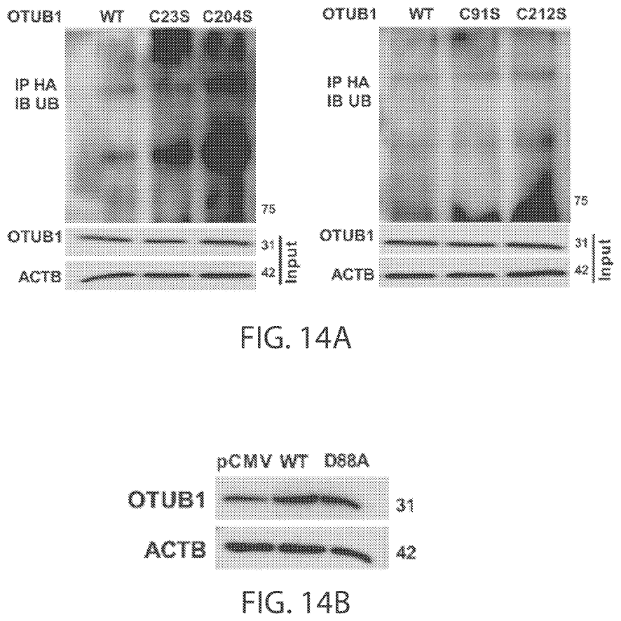

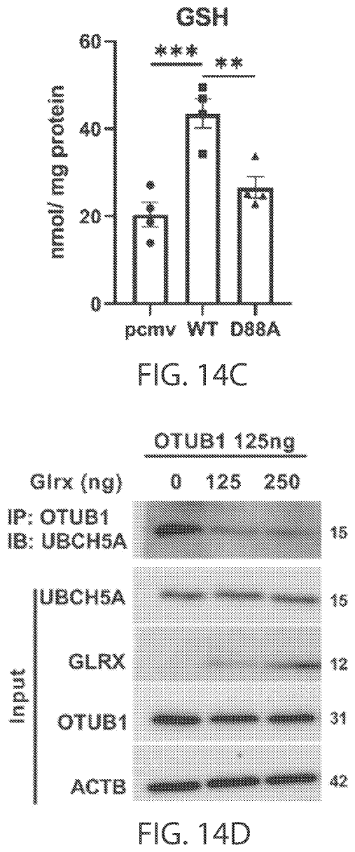

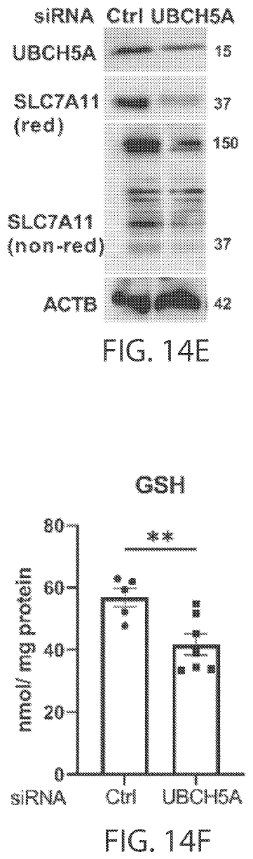

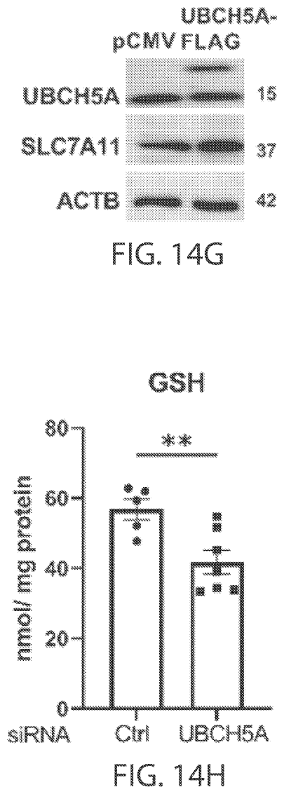

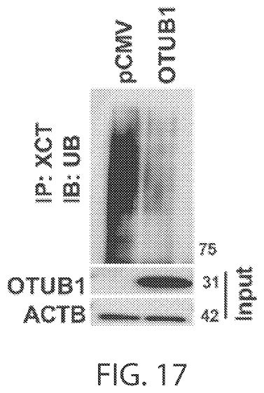

OTUB1 was previously shown to interfere with ubiquitination and subsequent proteasomal degradation of SLC7A11. Overexpression of OTUB1 decreased ubiquitinated SLC7A11 (FIG. 17). However, cells expressing OTUB1-C23S or OTUB1-C204S showed increased ubiquitination of SLC7A11 compared to cells expressing OTUB1-WT, OTUB1-C91S or OTUB1-C212S mutants (FIG. 14A). The OTUB1-mediated attenuation of ubiquitination can occur through multiple mechanisms. OTUB1 can act as a canonical deubiquitinase that cleaves K48 chains. Alternatively, OTUB1 also can inhibit the function of E2 conjugating enzymes, including UBCH5 and OTUB1 has been found in a complex with E2 enzymes. Additionally, binding of E2 enzymes to OTUB1 can also promote OTUB1 deubiquitinase activity dependent on the ratio of ubiquitin-charged E2 to uncharged E2 enzyme and levels of free ubiquitin. OTUB1 D88 binds E2 enzymes and suppresses Ub-conjugating activity. Stabilization of SLC7A11 by OTUB1 has been speculated to involve directly binding between OTUB1 and SLC7A11 as well as the inhibition of E2 enzymes. Consistent with this scenario, OTUB1 D88A failed to induce increases in GSH levels in H522 cells (FIGS. 14B-14C). These collective observations, along with observed increases in GSH following expression of OTUB1-C91S (FIG. 6C) support a non-canonical role of OTUB1 in augmenting GSH. Separate domains within OTUB1 are required for non-canonical inhibition via binding of ubiquitin-charged E2 enzyme (E2-Ub) and free ubiquitin (Ub), in a configuration that mimics the product of OTUB1-mediated deubiquitination. These domains in OTUB1 are in close proximity to C23 and C204, raising the possibility that S-glutathionylation at either site regulates binding or orientation of either free ubiquitin or E2-Ub enzyme. To address this possible scenario, the present disclosure determined whether OTUB1-S-glutathionylation affected the interaction between OTUB1 and UBCH5A which requires the N-terminal region containing C23. The previous data show that GLRX decreases OTUB1-SSG (FIG. 4L). GLRX also decreases the interaction between OTUB1 and UBCH5A (FIG. 14D). Knockdown of UBCH5A attenuated SLC7A11 levels and diminished GSH levels while conversely overexpression of UBCH5A augmented SLC7A11 and GSH (FIGS. 14E-14H).

### Example 10. Molecular Modeling Visualizing the Impact of OTUB1-SSG on the Binding Between OTUB1 and UBCH5A

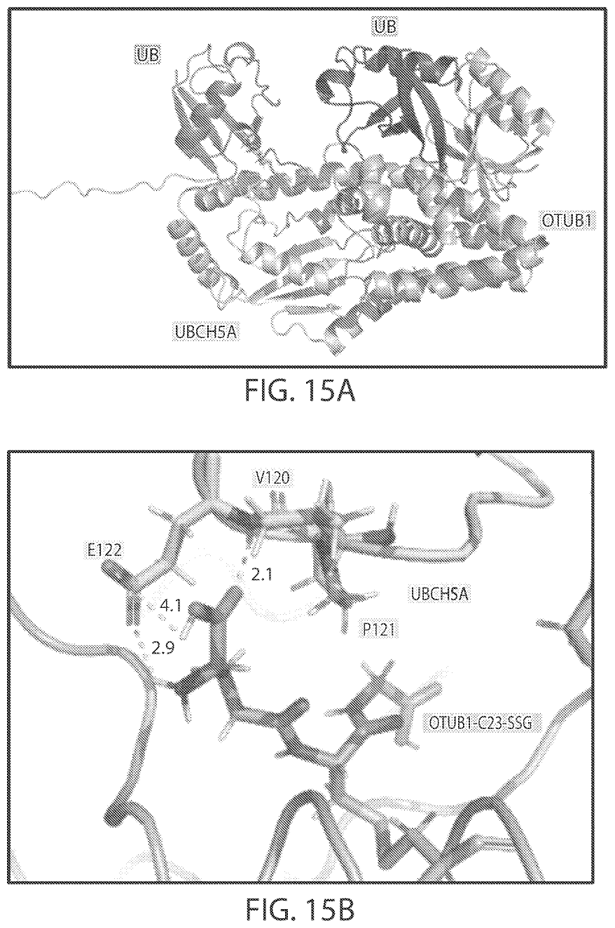

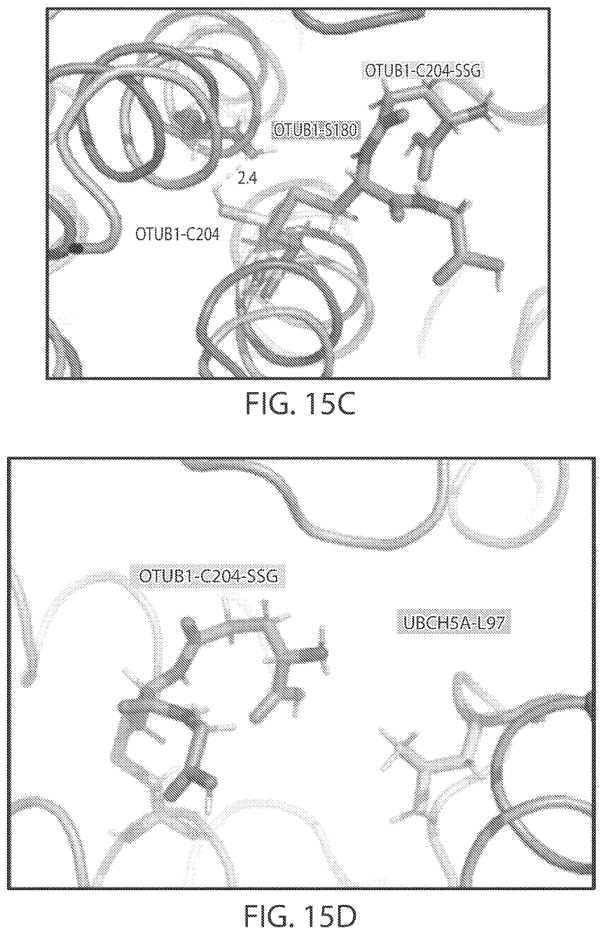

These findings suggest that UBCH5A augmented OTUB1's non-canonical activity through a mechanism that involves S-glutathionylation of OTUB1 at either position C23 or C204. To understand how S-glutathionylation affects the OTUB1-UBCH5A complex, the present disclosure created three different in silico models of OTUB1 in conjunction with Ub-charged UBCH5A and free Ub. In the first model, OTUB1 contains reduced cysteines while the other models consist of OTUB1 S-glutathionylated at C23 (OTUB1-C23-SSG) (FIGS. 15A-15B) or OTUB1 S-glutathionylated C204 (OTUB1-C204-SSG) (FIGS. 15C-15D). The overall root-mean-square deviation (RMSD) of UBCH5A was shown to remain locked into place regardless of the S-glutathionylation status of OTUB1. OTUB1-C23-SSG rapidly formed a stable contact between the terminal glutamic acid moiety of glutathione and a protein cleft at UBCH5A consisting of V120, P121, and E122 (FIG. 15B). Specifically, both the carbonyl and free amine of glutathione were shown to bind to the backbone amine and side chain carbonyl of E122 with contacts maintained under 3 Å (FIG. 15B). These contacts may serve to stabilize the protein-protein interaction of OTUB1-C23-SSG and UBCH5A when compared to reduced OTUB1. In contrast, OTUB1-C204-SSG was predicted to induce conformational changes in OTUB1 and to restrict the movement of C204 which is otherwise shown to oscillate towards Ser180 and form an internalized polar contact (FIG. 15C). Despite this change, no major stabilizing interactions were calculated between GSH and UBCH5A aside from the non-polar contact with L97 (FIG. 15D) and the site remained relatively isolated throughout simulation.

### Example 11. Decreases in GLRX and Increases in Overall PSSG and OTUB-SSG Occur in LUAD

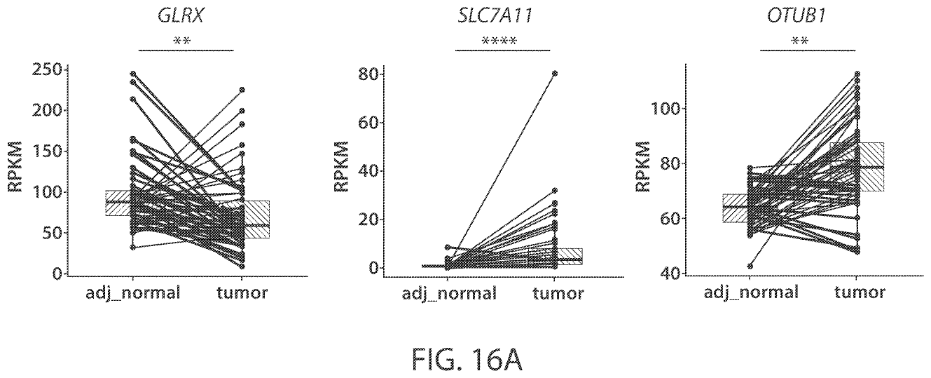

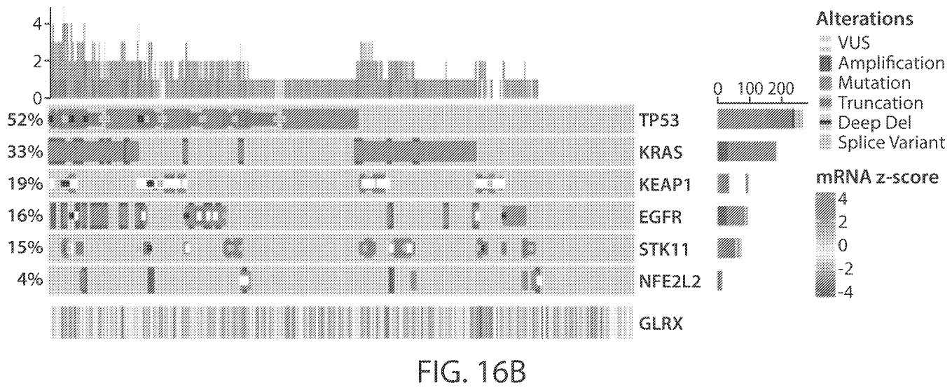

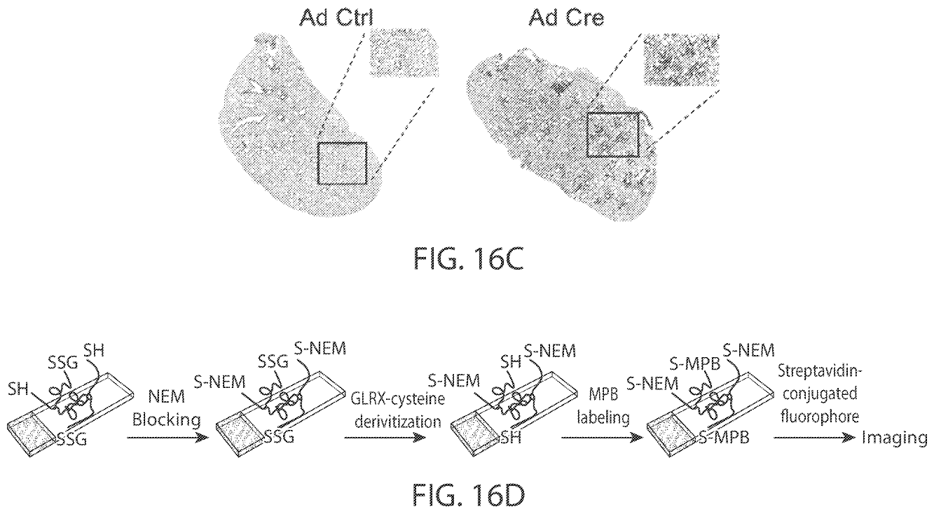

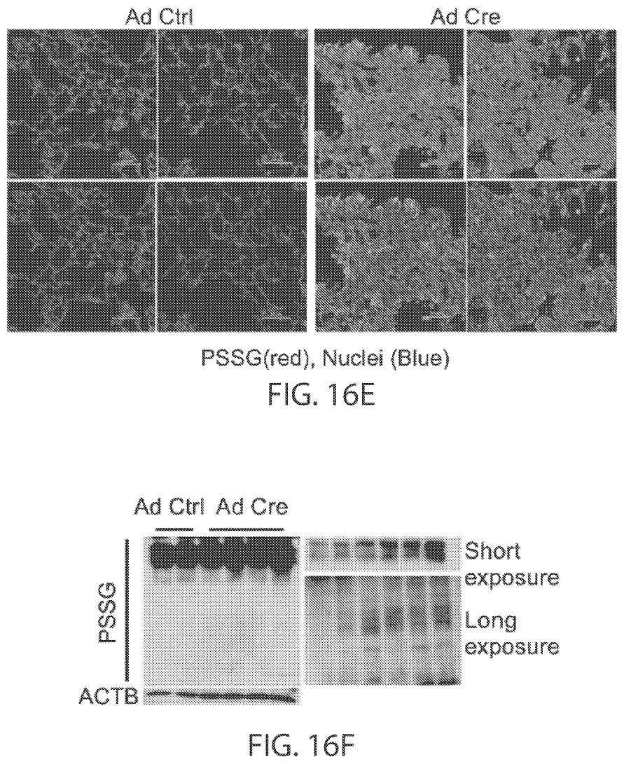

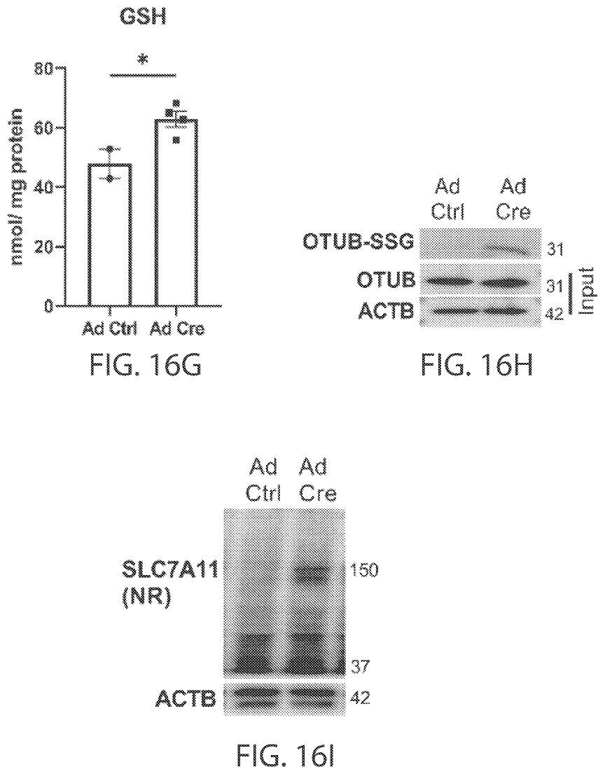

System X_{C}⁻ and OTUB1 have been extensively studied in cancer including LUAD. GLRX. SLC7A11 and OTUB1 expression in the Cancer Genome Atlas (TCGA) database was examined in order to address the potential relevance of GLRX, SLC7A11 and OTUB1 in LUAD. Evaluation of 58 LUADs with matched adjacent normal lung tissue, showed a decreased expression of GLRX in LUAD (FIG. 16A), while in contrast SLC7A11 and OTUB1 expression was increased relative to the adjacent normal tissue (FIG. 16A).

Visualizing GLRX expression and genomic alterations using Oncoprint revealed no clear association between GLRX and common LUAD oncogenic drivers (FIG. 16B). An adenovirus-expressing Cre-recombinase (AdCre) system was used to induce early tumors in Kras^{G12D} mice (FIG. 16C). GLRX-mediated cysteine derivatization was used to illuminate regions of PSSG, and showed increases in PSSG in early tumor regions compared to the control mice (FIGS. 16D-16E). Lastly, overall PSSG, GSH and OTUB-SSG increased in lungs with activated Kras^{G12D}-induced tumors, accompanied by increases in SLC7A11 protein levels (FIG. 16F-16I). The present disclosure did not detect changes in GLRX levels in the homogenized whole lung tissue, possibly due to the small representation of tumor area. Despite these different observations, the decreases in GLRX in LUAD and the increases in PSSG and OTUB1-SSG in KrasG12D-driven tumors in mice point to the relevance of findings herein for LUAD. These overall findings illuminate a mechanism of regulation of system xc through glutathione-dependent protein oxidation that is under OTUB1-dependent glutaredoxin-directed redox control (Graphical abstract) and highlight its putative relevance in lung cancer.

### OTHER EMBODIMENTS

U.S. Provisional Patent Application Ser. No. 63/388,203, filed Jul. 11, 2022, entitled “Ovarian Tumor Deubiquitinase Oxidation and Uses Thereof,” by Janssen-Heininger, et al., is incorporated herein by reference in its entirety.

In the claims articles such as “a.” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of treating a cancer, the method comprising administering an effective amount of an agent to a subject in need thereof, wherein the agent inhibits the oxidation under physiological conditions of a cysteine residue of ovarian tumor deubiquitinase (OTUB1).

2. The method of claim 1, wherein the cysteine residue of OTUB1 comprises Cys23.

3. The method of claim 1, wherein the cysteine residue of OTUB1 comprises Cys204.

4. The method of claim 1, wherein the agent inhibits the oxidation of a second cysteine residue of OTUB1.

5. The method of claim 4, wherein the agent inhibits the cysteine residue and the second cysteine residue comprise Cys23 and Cys204.

6. The method of any one of claims 1-5, wherein the cancer is selected from the group consisting of: breast cancer, ovarian cancer, lung cancer, liver cancer, colon cancer, glioma, melanoma, acute myeloid leukemia, esophageal cancer, gastric cancer, endometrial cancer, prostate cancer, and thyroid cancer.

7. The method of claim 6, wherein the lung cancer is selected from the group consisting of: non-small cell lung cancer, squamous cell carcinoma, adenocarcinoma, large-cell carcinoma and small-cell lung cancer.

8. The method of any one of claims 1-7, wherein the agent comprises a small molecule.

9. The method of any one of claims 1-7, wherein the agent comprises a glutaredoxin.

10. The method of claim 9, wherein the glutaredoxin is selected from the group consisting of glutaredoxin 1 (GRX1), GRX2, and GRX5.

11. The method of any one of claims 1-10, wherein the subject has a cancer.

12. The method of any one of claims 1-11, further comprising reducing a glutathione level in a cancer cell, destabilizing Solute Carrier Family 7 Member 11 (SLC7A11), decreasing the function of system xC- or any combination thereof.

13. The method of any one of claims 1-12, further comprising concomitantly administering an additional therapeutic agent.

14. The method of claim 13, wherein the additional therapeutic agent is an immune checkpoint inhibitor or a chemotherapeutic agent.

15. The method of claim 13, wherein the immune checkpoint inhibitor is selected from the group consisting of: CTLA4, PD1, PDL-1, B7H1, B7H3, B7H4, OX-40, CD137, CD40, CD27, LAG3, TIM3, ICOS, or BTLA.

16. The method of claim 14 or 15, wherein the chemotherapeutic agent comprises cisplatin.

17. An agent that inhibits the oxidation of at least one cysteine residue of ovarian tumor deubiquitinase (OTUB1) under physiological conditions.

18. The agent of claim 17, wherein the at least one cysteine residue of OTUB1 comprises Cys23.

19. The agent of claim 17, wherein the at least one cysteine residue of OTUB1 comprises Cys204.

20. The agent of any one of claims 17-19, wherein the at least one cysteine residue of OTUB1 comprises Cys23 and Cys204.

21. The agent of any one of claims 17-20, wherein the agent comprises a small molecule.

22. The agent of any one of claims 17-20, wherein the agent comprises a glutaredoxin.

23. The agent of claim 22, wherein the glutaredoxin comprises GRX1.

24. The agent of claim 22 or 23, wherein the gluaredoxin comprises GRX2.

25. The agent of any one of claims 22-24, wherein the glutaredoxin comprises GRX5.

26. A pharmaceutical composition comprising the agent of any one of claims 17-25 and a pharmaceutically acceptable excipient.

27. A kit comprising a container housing an agent that inhibits the oxidation of at least one cysteine residue of ovarian tumor deubiquitinase (OTUB1) and instructions for administering components in the kit to a subject having a cancer.

28. The kit of claim 27, further comprising a container housing a pharmaceutical preparation diluent.

29. The method of any one of claims 1-16, the agent of any one of claims 17-25, the pharmaceutical composition of claim 26, and the kit of any one of claims 27-28, wherein the agent is a non-covalent compound.

30. The method of any one of claims 1-16, the agent of any one of claims 17-25, the pharmaceutical composition of claim 26, and the kit of any one of claims 27-28, wherein the agent is a covalent compound.

31. The method of any one of claims 1-16, the agent of any one of claims 17-25, the pharmaceutical composition of claim 26, and the kit of any one of claims 27-28, wherein the agent comprises

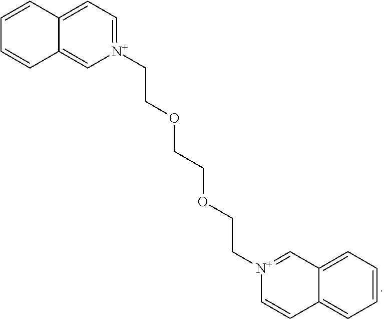 [SMILES: c1ccc2c[n+](CCOCCOCC[n+]3ccc4ccccc4c3)ccc2c1]

32. The method of any one of claims 1-16, the agent of any one of claims 17-25, the pharmaceutical composition of claim 26, and the kit of any one of claims 27-28, wherein the agent comprises

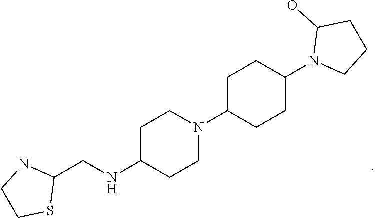 [SMILES: OC1CCCN1C1CCC(N2CCC(NCC3NCCS3)CC2)CC1]

33. The method of any one of claims 1-16, the agent of any one of claims 17-25, the pharmaceutical composition of claim 26, and the kit of any one of claims 27-28, wherein the agent comprises

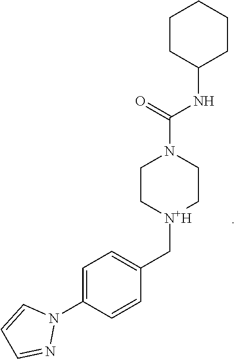

34. The method of any one of claims 1-16, the agent of any one of claims 17-25, the pharmaceutical composition of claim 26, and the kit of any one of claims 27-28, wherein the agent comprises

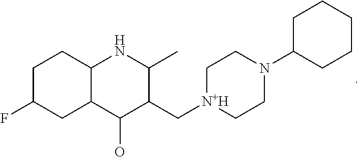

35. The method of any one of claims 1-16, the agent of any one of claims 17-25, the pharmaceutical composition of claim 26, and the kit of any one of claims 27-28, wherein the agent comprises

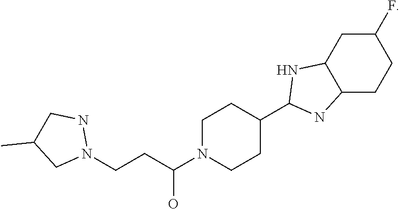 [SMILES: CC1CNN(CCC(O)N2CCC(C3NC4CCC(F)CC4N3)CC2)C1]

36. The method of any one of claims 1-16, the agent of any one of claims 17-25, the pharmaceutical composition of claim 26, and the kit of any one of claims 27-28, wherein the agent comprises 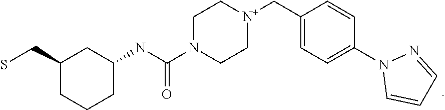 [SMILES: O=C(N[C@@H]1CCC[C@@H](CS)C1)N1CC[NH+](Cc2ccc(-n3cccn3)cc2)CC1]


SEQUENCE LISTING (16 sequences)

SEQ ID NO: 1 - DNA, 25 bp, synthetic construct
  source 1..25: mol_type = other DNA
caccgggatgtacgagtactttttg

SEQ ID NO: 2 - DNA, 25 bp, synthetic construct
  source 1..25: mol_type = other DNA
aaaccaaaaagtactcgtacatccc

SEQ ID NO: 3 - DNA, 25 bp, synthetic construct
  source 1..25: mol_type = other DNA
caccgatccgcaagaccaggcctga

SEQ ID NO: 4 - DNA, 25 bp, synthetic construct
  source 1..25: mol_type = other DNA
aaactcaggcctggtcttgcggatc

SEQ ID NO: 5 - DNA, 25 bp, synthetic construct
  source 1..25: mol_type = other DNA
caccgaggccagacagttaacacct

SEQ ID NO: 6 - DNA, 25 bp, synthetic construct
  source 1..25: mol_type = other DNA
aaacaggtgttaactgtctggcctc

SEQ ID NO: 7 - DNA, 35 bp, synthetic construct
  source 1..35: mol_type = other DNA
catcataggccagactgttaacaccttcggagtcg

SEQ ID NO: 8 - DNA, 35 bp, synthetic construct
  source 1..35: mol_type = other DNA
cgactccgaaggtgttaacagtctggcctatgatg

SEQ ID NO: 9 - DNA, 29 bp, synthetic construct
  source 1..29: mol_type = other DNA
agcccgatagaaactgttgccgtcaggcc

SEQ ID NO: 10 - DNA, 29 bp, synthetic construct
  source 1..29: mol_type = other DNA
ggcctgacggcaacagtttctatcgggct

SEQ ID NO: 11 - DNA, 29 bp, synthetic construct
  source 1..29: mol_type = other DNA
cacctcctgctggctgaactccttgacag

SEQ ID NO: 12 - DNA, 29 bp, synthetic construct
  source 1..29: mol_type = other DNA
ctgtcaaggagttcagccagcaggaggtg

SEQ ID NO: 13 - DNA, 27 bp, synthetic construct
  source 1..27: mol_type = other DNA
tcgctctccttgctcatgggctccacc

SEQ ID NO: 14 - DNA, 27 bp, synthetic construct
  source 1..27: mol_type = other DNA
ggtggagcccatgagcaaggagagcga

SEQ ID NO: 15 - protein, 271 aa, Homo sapiens
MAAEEPQQQKQEPLGSDSEGVNCLAYDEAIMAQQDRIQQEIAVQNPLVSERLELSVLYKEYAEDDNIYQQKIKDLHKKYSYIRKTRPDGNCFYRAFGFSHLEALLDDSKELQRFKAVSAKSKEDLVSQGFTEFTIEDFHNTFMDLIEQVEKQTSVADLLASFNDQSTSDYLVVYLRLLTSGYLQRESKFFEHFIEGGRTVKEFCQQEVEPMCKESDHIHIIALAQALSVSIQVEYMDRGEGGTTNPHIFPEGSEPKVYLLYRPGHYDILYK

SEQ ID NO: 16 - protein, 5 aa, synthetic construct
CPSYC